(19) United States
(12) Patent Application Publication
(10) Pub. No.: US 2024/0189293 A1
(43) Pub. Date: Jun. 13, 2024

(54) Compositions And Methods For Treating Gulf War Illness

(71) Applicant: The United States Government As Represented By The Department of Veterans Affairs, Washington, DC (US)

(72) Inventor: Bruce Citron, East Orange, NJ (US)

(21) Appl. No.: 18/347,811

(22) Filed: Jul. 6, 2023

Related U.S. Application Data
(60) Provisional application No. 63/358,624, filed on Jul. 6, 2022.

(51) Int. Cl. A61K 31/4439; A61K 31/05; A61K 45/06; A61P 25/28

(52) U.S. Cl. CPC A61K 31/4439; A61K 31/05; A61K 45/06; A61P 25/28

(57) ABSTRACT

The disclosure relates to compositions and methods of treating Gulf War illness or syndrome in a subject. The method comprises administering to a subject in need of treatment an effective amount of a nuclear receptor peroxisome proliferator-activated receptor gamma agonist and a nuclear factor erythroid 2-related factor 2 agonist.

### CROSS-REFERENCE TO RELATED APPLICATIONS

This application claims priority to and the benefit of the filing date of U.S. Provisional Application No. 63/358,624, filed Jul. 6, 2022, the entirety of which is incorporated by reference herein for all purposes.

### BACKGROUND

Gulf War Illness (GWI) is a chronic multi-system disorder affecting approximately 30% of Veterans deployed during Operations Desert Shield and Desert Storm from August 1990 to February 1991. GWI encompasses a wide spectrum of symptoms which typically include some combination of fatigue/sleep problems, pain, neurological/mood/cognitive impairments, respiratory complaints, gastrointestinal problems, or skin symptoms. Of particular interest are neurocognitive impairments and effects on the central nervous system (CNS), as Gulf War Veterans have significantly higher rates of neurological disorders, including amyotrophic lateral sclerosis (ALS), brain cancers, stroke, migraines, neuritis, and neuralgia, than other veteran populations. Therefore, an effective approach to prevent or manage GWI is needed.

### SUMMARY

Disclosed herein are methods of treating of Gulf War illness or syndrome in a subject, the methods comprising: administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist.

Disclosed herein are methods of ameliorating one or more symptoms of ameliorating one or more symptoms of Gulf War illness or syndrome in a subject, the methods comprising: administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist.

Disclosed herein are methods of inhibiting neurodegeneration or effecting neuroprotection in a subject in need thereof, the method comprising: administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist.

Disclosed herein are compositions comprising a nuclear receptor peroxisome proliferator-activated receptor gamma (PPAR-γ) agonist and a nuclear factor erythroid 2-related factor 2 (Nrf2) agonist.

Other features and advantages of the present compositions and methods are illustrated in the description below, the drawings, and the claims.

### BRIEF DESCRIPTION OF THE DRAWINGS

FIGS. 1A-B show that neuroprotective modulators result in improved behavioral performance long after model Gulf War insults. Gulf War exposure was modeled by 2 weeks of daily subcutaneous injections of pyridostigmine bromide (2.5 mg/kg), chlorpyrifos (12.5 mg/kg), and DEET (7.5 mg/kg). The injections began at 3 months of age. Sham animals received just the solvent solution without any of the toxicants. At 42 weeks of age (28 weeks after the last toxicant exposure) there were 2 weeks of daily ip injections of tBHQ (33.4 mg/kg) and pioglitazone (3.0 mg/kg) or vehicle. Behavioral testing was conducted by home cage monitoring in a PhenoMaster system. FIG. 1A shows that the total distance traveled in a running wheel over 10 days beginning 3 days after the treatment was completed indicates that the toxicant exposure resulted in reduced participation in the running wheel and this reduction did not appear in the group that had received the treatment. FIG. 1B shows that by measuring the x-y movements in the home cage (monitored with infrared beams), the Gulf War insult caused a large reduction in exploratory behavior during the active, nocturnal period measured at 2 weeks after the last treatment. This reduction disappeared in the GWI mice that received the neuroprotective modulator treatment.

FIG. 2 shows the RNA-Seq analysis workflow with CLC Genomics Workbench and Ingenuity Pathway Analysis. Whole transcriptome sequencing was performed using mouse hippocampal RNA isolates collected 2-4 hours after final exposure. Gene expression tracks were analyzed using the Differential Expression for RNA-Seq tool with RPKM>10.0, |FC|≥1.2, and p<0.1 as criteria for significance. GO enrichment analysis was performed on subset of genes that were significantly dysregulated. Data for significant genes was exported to Ingenuity Pathway Analysis to assess canonical pathways, molecules, diseases and functions, and other relevant information.

FIGS. 3A-C shows (FIG. 3A) Preference for novel arm, (FIG. 3B) number of entries per arm, and (FIG. 3C) distance travelled during trial phase of Y-maze. Hippocampal-dependent spatial memory was assessed by performance on a Y-maze task 2-4 hours after final exposure. FIG. 3A shows the preference for the novel arm was significantly lower in mice receiving PB+CPF+DEET (mean=−0.12±0.099) compared to control mice (mean=0.21±0.073) (t(9.18)=2.63, p=0.027). FIG. 3B shows the number of entries into the novel arm was also significantly lower in mice exposed to PB+CPF+DEET (mean=15.2±1.15) compared to controls (mean=22.7±1.52) (t(9.31)=3.95, p=0.0031). FIG. 3C shows the distance travelled during the test stage did not significantly differ between conditions (PB+CPF+DEET: mean=26.6±2.32, control: mean=30.2±0.74, t(6.00)=1.51, p=0.18). All results are graphed as mean±SEM.

FIG. 4 shows the differentially expressed genes identified by RNA-Seq analysis. Sequence counts from the RNA samples were evaluated with CLC Genomics Workbench and Ingenuity Pathway Analysis software. 158 dysregulated genes were identified in mice exposed to PB+CPF+DEET vs. controls. Genes were considered to be significantly dysregulated if they met the following criteria: RPKM>10.0, |fold change|≥1.2, p<0.1.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for Gulf War illness or syndrome, such as, for example, prior to the administering step.

As used herein, the term “treat” or “treatment” refers to the medical management of a patient with the intent to cure, ameliorate, stabilize, (e.g., Gulf War illness or syndrome). This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) inhibiting the disease, i.e., arresting its development; or (ii) relieving the disease, i.e., causing regression of the disease (e.g., Gulf War illness or syndrome).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, “prevent” is meant to mean minimize the chance that a subject who has an increased susceptibility for developing Gulf War illness or syndrome will develop Gulf War illness or syndrome. In the context as used herein, preventing does not need to eliminate completely all sequel associated with Gulf War illness or syndrome and would encompass any reduction in the expression of one or more symptoms associated or disease conditions associated with Gulf War illness or syndrome.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, or affection.

“Inhibit,” “inhibiting” and “inhibition” mean to diminish or decrease gene expression, activity, response, condition, disease, or other biological parameter. This can include, but is not limited to, the complete ablation of the activity, response, condition, or disease. This may also include, for example, a 10% inhibition or reduction in gene expression, activity, response, condition, or disease as compared to the wild-type or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to wild-type or control levels. In some aspects, the inhibition or reduction is 0-25, 25-50, 50-75, or 75-100% as compared to wild-type or control levels.

The terms “reducing”, “inhibiting” and “ameliorating” as used herein, when used in the context of modulating a pathological or disease state, generally refers to the prevention and/or reduction of at least a portion of the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

Disclosed herein are compositions comprising a nuclear receptor peroxisome proliferator-activated receptor gamma (PPAR-γ) agonist and a nuclear factor erythroid 2-related factor 2 (Nrf2) agonist that are useful in treating Gulf War illness or syndrome in subjects. The combination of a PPAR-γ agonist and a Nrf2 agonist is important because approximately one third of the 700,000 service personnel deployed during Operations Desert Storm and Desert Shield have developed Gulf War Illness and many of these Veterans suffer learning and memory impairments, sleep disturbances, neurodegeneration or a combination thereof. No effective treatments for Gulf War illness are available. The compositions disclosed herein may also be useful for treating other neurodegenerative conditions because there is increased recognition of widespread toxic exposures beyond the Gulf War.

While traumatic brain injuries (TBI) may be associated with learning or memory impairments and other symptoms, traumatic brain injuries are distinct from the effects of Gulf War illness because traumatic brain injuries involve a mechanical insult that typically results in some amount of necrosis or immediate cell damage and loss plus a secondary injury in a penumbra, surrounding any localized damage, due to activation of cell death cascades, blood brain barrier disruption, and ischemic responses. Gulf War illness involves exposure of the entire brain to the toxicants without any local necrosis or the TBI types of cell death. Thus, compositions and treatments that may be useful for treating or TBI do not predict efficacy in other diseases, illnesses, syndromes or disorders of the brain.

### Compositions

Disclosed herein are compositions comprising a nuclear receptor peroxisome proliferator-activated receptor gamma (PPAR-γ) agonist and a nuclear factor erythroid 2-related factor 2 (Nrf2) agonist.

In some aspects, the nuclear receptor peroxisome proliferator-activated receptor gamma (PPAR-γ) agonist and a nuclear factor erythroid 2-related factor 2 (Nrf2) agonist are co-formulated. In some aspects, the nuclear receptor peroxisome proliferator-activated receptor gamma (PPAR-γ) agonist and a nuclear factor erythroid 2-related factor 2 (Nrf2) agonist are co-packaged.

In some aspects, the nuclear receptor peroxisome proliferator-activated receptor gamma (PPAR-γ) agonist and a nuclear factor erythroid 2-related factor 2 (Nrf2) agonist can be present in a therapeutically effective amount for treating or ameliorating one or more symptoms of Gulf War illness or syndrome in a subject.

In some aspects, the PPAR-γ agonist can be pioglitazone. In some aspects, the PPAR-γ agonist can be rosiglitazone.

In some aspects, the Nrf2 agonist can be tert-butylhydroquinone (t-BHQ). In some aspects, the Nrf2 agonist can be sulforaphane.

The compositions described herein can be formulated to include a therapeutically effective amount of a PPAR-γ agonist and a Nrf2 agonist described herein. The compositions described herein can be formulation in a variety of combinations. In some aspects, the composition can comprise pioglitazone and t-BHQ. In some aspects, the composition can comprise pioglitazone and Sulforaphane. In some aspects, the composition can comprise rosiglitazone and tBHQ. In some aspects, the composition can comprise rosiglitazone and Sulforaphane. The particular combination can vary according to many factors, for example, the particular the type and severity of the Gulf War illness or syndrome.

### Methods of Treatment

Disclosed herein are methods of treating of Gulf War illness or syndrome in a subject. In some aspects, the methods can comprise administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist. In some aspects, the administration of a PPAR-γ agonist and a Nrf2 agonist can reduce or ameliorate one or more symptoms of Gulf War illness or syndrome. In some aspects, the one or more symptoms of Gulf War illness or syndrome can be fatigue, musculoskeletal pain, skin rashes, diarrhea, headache, memory loss, spatial memory deficits, sleep disturbances or a combination thereof. Examples of sleep disturbances can include but are not limited to shorter bouts of rest.

Also disclosed herein are methods of ameliorating one or more symptoms of ameliorating one or more symptoms of Gulf War illness or syndrome in a subject. In some aspects, the methods can comprise administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist. In some aspects, the one or more symptoms of Gulf War illness or syndrome can be fatigue, musculoskeletal pain, skin rashes, diarrhea, headache, memory loss, spatial memory deficits, sleep disturbances or a combination thereof. Examples of sleep disturbances can include but are not limited to shorter bouts of rest.

In some aspects, the one or more symptoms of Gulf War illness or syndrome can be acute or chronic. In some aspects, one or more symptoms of Gulf War illness or syndrome or Gulf War illness or syndrome can be acute or in an acute phase. In some aspects, “acute” can mean that the one or more symptoms Gulf War illness or syndrome can appear in a period of time immediately after an exposure to one or more toxicants, and can last one day, one week, one month, two months, three months, four months, five months or less than six months.

In some aspects, one or more symptoms of Gulf War illness or syndrome Gulf War illness or syndrome can be chronic or in a chronic phase. In some aspects, “chronic” can mean that the one or more symptoms Gulf War illness or syndrome can appear about six months, seven months, eight months, nine months, ten months, eleven months, one year or more after an exposure to one or more toxicants.

Further disclosed herein are methods of inhibiting neurodegeneration or effecting neuroprotection in a subject in need thereof. In some aspects, the methods can comprise administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist. In some aspects, the neurodegeneration or neuroprotection can be associated with Alzheimer's disease, Parkinson's disease, traumatic brain injury, amyotrophic lateral sclerosis, ischemic stroke or a combination thereof. In some aspects, neuroprotection, or reduced or inhibited neurodegeneration, can be indicated by improved outcomes in terms of behavioral performance, e.g., restoration of exploratory behavior, and reduced neuropathology.

In some aspects, any of the methods disclosed herein can increase stamina, and/or improve cognition and information seeking in a subject.

In some aspects, the subject has been diagnosed with Gulf War illness prior to the administering step. In some aspects, the subject has been exposed to one or more Gulf War toxicants. In some aspects, the one or more Gulf War toxicants can be pyridostigmine bromide, chlorpyrifos, or N,N-diethyl-m-toluamide (DEET). In some aspects, the one or more Gulf War toxicants can be an anti-sarin prophylactic, an organophosphate insecticide, or an insect repellant. In some aspects, the one or more Gulf War toxicants can be sarin, burn pit exposures, depleted uranium, chemical agent resistant coatings, and other pesticides.

In some aspects, the PPAR-γ agonist can be pioglitazone. In some aspects, the PPAR-γ agonist can be rosiglitazone.

In some aspects, the Nrf2 agonist can be tert-butylhydroquinone (t-BHQ). In some aspects, the Nrf2 agonist can be sulforaphane.

The compositions described herein can be formulated to include a therapeutically effective amount of a PPAR-γ agonist and a Nrf2 agonist described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to Gulf War illness or syndrome.

The compositions described herein can be formulation in a variety of combinations. In some aspects, the composition can comprise pioglitazone and t-BHQ. In some aspects, the composition can comprise pioglitazone and Sulforaphane. In some aspects, the composition can comprise rosiglitazone and tBHQ. In some aspects, the composition can comprise rosiglitazone and Sulforaphane. The particular combination can vary according to many factors, for example, the particular the type and severity of the Gulf War illness or syndrome.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with Gulf War illness or syndrome in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the Gulf War illness or syndrome is delayed, hindered, or prevented, or the Gulf War illness or syndrome or a symptom of the Gulf War illness or syndrome is ameliorated. One or more of the symptoms can be less severe. Recovery can be accelerated in an individual who has been treated.

The compositions described herein can be formulated to include a therapeutically effective amount of a PPAR-γ agonist and a Nrf2 agonist. In some aspects, the PPAR-γ agonist and the Nrf2 agonist can be contained within the same or within a separate pharmaceutical formulation. In some aspects, the pharmaceutical formulation can be a unit dosage formulation.

The therapeutically effective amount or dosage of any of the PPAR-γ agonists and any of the Nrf2 agonists used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the Gulf War illness or syndrome symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Dosages of pioglitazone can be in the range of 0.1 mg to 0.4 mg/kg body weight per day. In some aspects, the dosage of pioglitazone can be 0.1, 0.15, 0.2, 0.25, 0.3, 0.35 or 0.4 mg/kg total or any amount in between. In some aspects, the therapeutically effective dose of pioglitazone can be less when combined with the Nrf2 agonist disclosed herein.

Dosages of rosiglitazone can be in the range of 0.1 mg to 0.4 mg/kg body weight per day. In some aspects, the dosage of rosiglitazone can be 0.1, 0.15, 0.2, 0.25, 0.3, 0.35 or 0.4 mg/kg total or any amount in between. In some aspects, the therapeutically effective dose of rosiglitazone can be less when combined with the Nrf2 agonist disclosed herein.

Dosages of tert-butylhydroquinone can be in the range of 1.0 mg to 5.0 mg/kg body weight per day. In some aspects, the dosage of tert-butylhydroquinone can be 1.0, 1.5, 2.0, 2.5, 3.0, 3.5, 4.0, 4.5 or 5.0 mg/kg total or any amount in between.

Dosages of sulforaphane can be in the range of 0.15 mg to 0.7 mg/kg body weight per day. In some aspects, the dosage of sulforaphane can be 1.5, 2.0, 2.5, 3.0, 3.5, 4.0, 4.5, 5.0, or 8 mg/kg total or any amount in between.

Suitable treatment regimens using any of the dosages described herein include, but are not limited to: any of the PPAR-γ agonists and any of the Nrf2 agonists daily once; any of the PPAR-γ agonists and any of the Nrf2 agonists once a week; any of the PPAR-γ agonists daily once and any of the Nrf2 agonists once a week; any of the PPAR-γ agonists once a week and any of the Nrf2 agonists daily once.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes. For example, a PPAR-γ agonist and a Nrf2 agonist can be administered in conjunction with standard therapies used to treat neurodegenerative diseases or disorders. In some aspects, a PPAR-γ agonist and a Nrf2 agonist or any of the compositions described herein can be administered or used together with one or more transcription factor modulators. In some aspects, the PPAR-γ agonist and the Nrf2 agonist are co-formulated. In some aspects, pioglitazone and t-BHQ are co-formulated. In some aspects, pioglitazone and Sulforaphane are co-formulated. In some aspects, rosiglitazone and tBHQ are co-formulated. In some aspects, rosiglitazone and Sulforaphane are co-formulated.

Any of the compounds or compositions described herein can be administered as a term “combination.” It is to be understood that, for example, PPAR-γ agonist can be provided to the subject in need, either prior to administration of the Nrf2 agonist, concomitant with administration of the Nrf2 agonist (co-administration) or shortly thereafter.

### Pharmaceutical Compositions

As disclosed herein, are pharmaceutical compositions, comprising one or more of the therapeutic compositions or inhibitors disclosed herein. As disclosed herein, are pharmaceutical compositions, comprising a PPAR-γ agonist and a Nrf2 agonist and a pharmaceutical acceptable carrier described herein. In some aspects, the PPAR-γ agonist and the Nrf2 agonist can be formulated for oral or parenteral administration. In some aspects, the parenteral administration can intravenous, subcutaneous, intraperitoneal, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes a PPAR-γ agonist and a Nrf2 agonist dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In some aspects, a pharmaceutical composition comprises a) a PPAR-γ agonist and a Nrf2 agonist; and b) optionally, a pharmaceutical acceptable carrier. Further, the pharmaceutical composition comprises a PPAR-γ agonist and a Nrf2 agonist in therapeutically effective amounts. In some aspects, the PPAR-γ agonist can be pioglitazone or rosiglitazone. In some aspects, the Nrf2 agonist can be tert-butylhydroquinone (t-BHQ) or sulforaphane. In some aspects, the pharmaceutical composition can be formulated for oral, intravenous, intraperitoneal or subcutaneous administration.

### Articles of Manufacture

The compositions described herein can be packaged in a suitable container labeled, for example, for use as a therapy to treat Gulf War illness or syndrome or any of the methods disclosed herein. Accordingly, packaged products (e.g., sterile containers containing the composition described herein and packaged for storage, shipment, or sale at concentrated or ready-to-use concentrations) and kits, including at least a PPAR-γ agonist and a Nrf2 agonist and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compounds can be provided in a concentrated form with a diluent and instructions for dilution.

In some aspects, the PPAR-γ agonist and the Nrf2 agonist can be co-packaged. In some aspects, the pioglitazone and t-BHQ can be co-packaged. In some aspects, pioglitazone and Sulforaphane can be co-packaged. In some aspects, rosiglitazone and tBHQ can be co-packaged. In some aspects, rosiglitazone and Sulforaphane can be co-packaged.

### EXAMPLES

### Example 1: Neuroprotection for Gulf War Illness Chronic Phase

The results described herein demonstrate the effectiveness for the neuroprotective modulators (tBHQ+pioglitazone) in the Gulf War illness model in the chronic phase. These data are important because veterans suffering Gulf War illness are in their chronic phases and this has significant implications for other neurodegenerative disorders. Acute phases can correspond to the period of time immediately after the exposure. Chronic phases can involve effects presenting greater than, for example, one year to anytime longer after exposure.

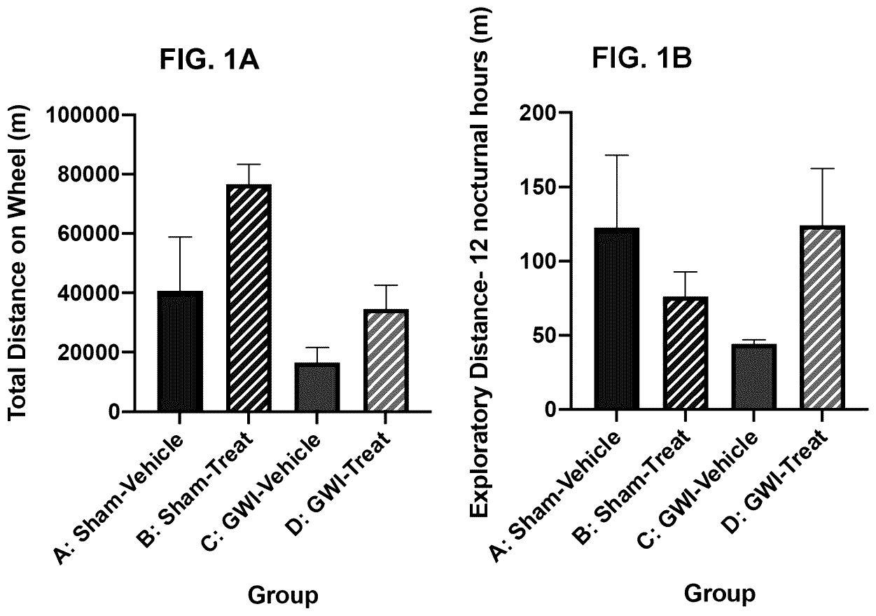

An established model of Gulf War illness was used for these studies (K. E. Murray, Life Sci. 284 (2021)). Gulf War model neurodegeneration was induced by a two week course of simultaneous administration of pyridostigmine bromide, chlorpyrifos, and DEET by daily subcutaneous injections corresponding to the common combined exposure of the anti-sarin prophylactic, organophosphate insecticide, and insect repellant, respectively, that deployed service personnel are exposed. The toxicants were administered to mice at 3 months of age. As shown in the FIG. 1, treatment late in the chronic phase improved behavioral performance values at 10 months of age, equivalent to middle age for the C57Bl/6 model mice.

In sum, the data show that the increased distance on the wheel indicates an increased stamina while the increased exploratory distance indicates improved cognition and information seeking in subjects receiving an administration of tBHQ and pioglitazone.

### Example 2: Treatment-Induced Improvements with Different Dosing and Timing after Changes in CNS Connectivity Induced by Exposure to Gulf War Toxicants

Neuropathology will be examined by immunohistochemistry, to observe region and cell specific changes in signaling factors, and also by Golgi staining, to determine dendritic complexity and spine density.

Because chronic effects in the brain have been observed due to Gulf War exposure-induced neuroplasticity changes, the improvements that are achievable with different combinations of transcription factor modulators (e.g., SP600125, pifithrin derivatives) will be determined. Groups will be tested for the matrix of vehicle vs. toxicants, control vs. treated, and male vs. female. Golgi staining will be performed to evaluate dendrites and spines. Different brain regions including the prefrontal cortex, perirhinal cortex, hippocampus, and the pontine reticular nucleus will also be evaluated.

### Example 3: Evaluate Functional Outcomes in a Gulf War Exposure Model with and Without Treatment

Cognitive tests will be performed with using the Gulf War illness mouse model, and compared to reported observations from Gulf War veterans to further assess similarities and differences between the model system and Gulf War veterans.

The functional consequences will be determined to better understand the effects of these kinds of exposures and the brain regions most affected by the treatment. Improved understanding of these processes will help characterize the effects of this treatment. For example, the animal groups will include the matrix of vehicle vs. Gulf War exposure, control vs. treated, and male vs. female. Behavioral tests will include novel object placement, Y maze, elevated plus maze testing, non-associative learning and impulse control.

### Example 4: Identify Neuroprotective Pathway Changes within the Central Nervous System (CNS) Affected by the Toxicant Exposure and Treatment

Changes at the protein and mRNA levels will be evaluated for the specific factors involved in the Gulf War exposure induced neurodegeneration that are accomplished by the treatment. For example, factors important to the health of neurons in the central nervous system such as inflammatory response elements will be assessed.

No treatment is available for neurodegeneration that manifests after Gulf War exposures. However, benefits of a combination of neuroprotective modulators (e.g., a nuclear receptor peroxisome proliferator-activated receptor gamma (PPAR-γ) agonists and a nuclear factor erythroid 2-related factor 2 (Nrf2) agonists) on long-term outcomes after Gulf War exposures have not been evaluated particularly with respect to inflammation and anti-inflammatory pathways. Many gene expression changes caused by Gulf War exposure result in levels that are detrimental to the optimal function of neurons, e.g., downregulation of Arc reducing neuroplasticity.

The matrix of vehicle vs. Gulf War toxicants, vehicle vs. combined (tBHQ, pioglitazone) treatment and male vs. female will be tested. Brain regions will be analyzed for molecular differences in inflammatory factor signaling and other dysregulations and modulations.

### Example 5: Acute Gene Expression Changes in the Mouse Hippocampus Following a Combined Gulf War Toxicant Exposure

Research findings in Gulf War animal models have demonstrated that a wide array of physiological alterations including changes in behavior, cognition, neurotransmission, axonal transport, genomic, proteomic, lipidomic, and metabolomic profiling, and mitochondrial dysfunction result from Gulf War exposure (Institute of Medicine, Gulf War and Health: Volume 8: Update of Health Effects of Serving in the Gulf War, Washington, D.C., 2010; Institute of Medicine, Chronic Multisymptom Illness in Gulf War Veterans: Case Definitions Reexamined, Washington, D.C., 2014; United States Department of Veterans Affairs, Research Advisory Committee on Gulf War Veterans' Illnesses [RAC-GWI], Gulf War illness and the health of Gulf War veterans: scientific findings and recommendations, Washington, D.C., 2008; Institute of Medicine, Gulf War Veterans: Treating Symptoms and Syndromes, Washington, D.C., 2001; Institute of Medicine, Gulf War and Health: Treatment for Chronic Multisymptom Illness, Washington, D.C., 2013; R. F. White, et al., Cortex 74 (2016) 449-475; and B. Dickey, et al., Pharmacol Ther (2020) 107716).

Military personnel deployed during the Gulf War were exposed to an array of chemical exposures in tandem, particularly acetylcholinesterase (AChE) inhibitors. Investigations into the effects of combined Gulf War exposures vary widely but typically include some combination of insecticides, insect repellants, nerve agents, and anti-toxins against nerve agents (Institute of Medicine, Gulf War and Health: Volume 8: Update of Health Effects of Serving in the Gulf War, Washington, D.C., 2010; Institute of Medicine, Chronic Multisymptom Illness in Gulf War Veterans: Case Definitions Reexamined, Washington, D.C., 2014; United States Department of Veterans Affairs, Research Advisory Committee on Gulf War Veterans' Illnesses [RAC-GWI], Gulf War illness and the health of Gulf War veterans: scientific findings and recommendations, Washington, D.C., 2008; and R. F. White, et al., Cortex 74 (2016) 449-475). The Gulf War toxicant mixture used in this Example includes chemicals from three of the most frequently investigated of these classes: specifically, pyridostigmine bromide (PB, a reversible AChE inhibitor administered as a sarin prophylactic), chlorpyrifos (CPF, an organophosphate pesticide), and N,N-diethyl-m-toluamide (DEET, a common insect repellent).

Significant pathological changes in the hippocampus and corresponding impairments in hippocampal-dependent learning and memory have been observed in several animal models of Gulf War toxicant exposure. Rats exposed to low doses of DEET, permethrin, PB, and restraint stress for four weeks showed significantly reduced hippocampal volume and neuron growth as well as increased occurrence of activated microglia and astrocyte hypertrophy which was accompanied by spatial learning and memory dysfunction (V. K. Parihar, et al., Neuropsychopharmacology 38 (2013) 2348-2362). The combination of PB and DEET has been shown to influence cholinesterase activity in the brain and affect seizures (L. A. Chaney, et al., Toxicol Sci 49 (1999) 306-311; and L. A. Chaney, et al., Toxicol Appl Pharmacol 165 (2000) 107-114). Organophosphate exposure has also been shown to impair spatial navigation learning in the Morris Water Maze task (M. A. Prendergast, et al., Psychopharmacology (Berl) 129 (1997) 183-191; and A. V. Terry, Jr., et al., J Pharmacol Exp Ther 305 (2003) 375-384). Neurotoxicity following administration of PB+CPF+DEET was originally reported by Abou-Donia et al. in hens exposed to 5 mg/kg PB i.o., 10 mg/kg CPF s.c., and 500 mg/kg DEET s.c. 5 days/week for 2 months (M. B. Abou-Donia, et al., Fundam Appl Toxicol 34 (1996) 201-222). Ojo et al. reported significant pathological changes in the hippocampus and cortex of C57Bl/6 mice exposed to PB+CPF+permethrin at an acute timepoint (72 hours post-exposure) (J. O. Ojo, et al., Neuropathology 34 (2014) 109-127).

Transcriptional changes after Gulf War toxicant exposure in rodent models have mostly focused on epigenetic changes or investigation of specific gene categories of interest at chronic timepoints (B. Dickey, et al. Pharmacol Ther (2020) 107716; G. A. Shetty, et al., Front Mol Neurosci 10 (2017) 182; L. M. Pierce, et al., Neurotoxicology 55 (2016) 20-32; D. G. Ashbrook, et al., J Neuroinflammation 15 (2018) 86; and F. Xu, et al., Brain Behav Immun 89 (2020) 209-223). Acute changes in gene expression were assessed in mouse hippocampal RNA isolates after exposure to a combined subcutaneous (s.c.) injection of PB, CPF, and DEET for two weeks using whole transcriptome sequencing (RNA-Seq). Genes important for neuronal health, those that could affected by toxicants, and genes involved in inflammatory responses were the focus. Differentially expressed genes observed at an acute timepoint may set the stage for chronic outcomes and may provide insight into the pathophysiology of Gulf War Illness and help identify targets for treatment.

Materials and methods. Chemicals. HPLC-grade pyridostigmine bromide (PB, P9797) and N,N-diethyl-m-toluamide (DEET, D100951) were obtained from Sigma-Aldrich (St. Louis, MO). Chlorpyrifos (CPF, N-11459) was obtained from ChemService, Inc. (West Chester, PA). The toxicant mixture stock was prepared and stored in 500 μL aliquots at −20° C. until use and diluted in PBS immediately prior to injection. Vehicle for injection contained 3.125% dimethyl sulfoxide (DMSO, 99.9%, D2438-5X10ML) obtained from Thermo Fisher Scientific (Waltham, MA) in 1×PBS.

Subjects. Animals were single-housed in a 22° C.±0.5° C. temperature-controlled environment with a 12-hour light/dark cycle. Animals were allowed a 7-day acclimation period before switching to a reverse light cycle (i.e., dark cycle from 10 am-10 pm) for 5 days prior to exposure. Food and water were available ad libitum throughout for the animals.

Toxicant exposure. Male C57Bl/6J mice were obtained from Charles River (Wilmington, MA) for RNA-seq (n=6/group) and from Jackson Laboratory (Bar Harbor, ME) for behavior (n=6/group) based on availability. Mice received daily s.c. injections of either the toxicant mixture containing 2.5 mg/kg PB, 12.5 mg/kg CPF, and 7.5 mg/kg DEET in PBS or vehicle containing 3.125% DMSO in PBS five days a week (M-F) for two weeks beginning at 12 weeks of age. Adverse effects including seizures resulting in removal and euthanasia, were observed at 1.5- and 2.0-fold higher dosages, but this was rare at the dosage used in this study. Experimental cohorts which generated RNA-seq and behavioral data did not display any significant adverse effects. For RNA-Seq, mice were sacrificed 2-4 hours after the final exposure via cervical dislocation and decapitation. Whole brains were immediately extracted, and hippocampal tissue from each hemisphere was dissected and snap frozen on dry ice. The fresh frozen tissue samples were stored at −70° C. until use.

Y-maze task with preference index. To assess hippocampal-dependent memory, subjects underwent a modified Y-maze task 2-4 hours after the final exposure. During the training phase, either Arm B or C (novel arm) was blocked off with a barrier. The novel arm was randomly assigned for each trial. Mice were placed in the start arm (Arm A) of the Y-maze facing the wall and allowed to explore the start and familiar arms for 8 minutes. Mice were then removed from the maze and returned to their home cage for an inter-trial interval of 30 minutes. During the trial phase, the barrier was removed so that all three arms were accessible. Mice were again placed in the start arm and allowed to explore the start and familiar arms for 8 minutes. Behavior was captured with a video camera (DMK 22AUC03, The Imaging Source, Charlotte, NC) and recorded by ANY-maze (Version 6.17, Stoelting, Wood Dale, IL). Time or entry into a zone was scored based on the center point of the animal's body. The Y-maze trials were performed under red light during the dark cycle.

RNA isolation. Hippocampal RNA was isolated by TRIzol (Invitrogen, Waltham, MA) extraction followed by cleanup with a RNeasy Mini Kit (QIAGEN, Hilden, Germany). Tissue was resuspended in 0.4 mL TRIzol and homogenized with a Polytron homogenizer (Kinematica USA, Bohemia, NY) on ice for 30-45 seconds. Samples were incubated at 23° C. for 5 minutes before adding 80 μL CHCl₃ and vortexing for 15 seconds. Samples were incubated at 23° C. again for 2-3 minutes. Tubes were centrifuged at 12,000 rcf for 10 minutes, and the supernatant was transferred into a new tube with an equal volume of 70% EtOH. RNeasy Mini Kit was then used per the manufacturer's instructions with Tris-EDTA buffer (TE, pH 8.0, AM9858, Invitrogen) for the final elution step. All RNA isolates were stored at −20° C. until use.

RNA-Seq. RNA isolates were sequenced. Total cellular RNA was qualified by confirming integrity with a 2200 TapeStation (Agilent Technologies, Santa Clara, CA). Samples with an RNA integrity number (RIN)>7.0 were used for subsequent processing. Total RNAs were subjected to two rounds of poly(A) selection using Oligo d(T)₂₅ Magnetic Beads (New England Biolabs, Ipswich, MA). RNA-Seq libraries were prepared using an NEBNext Ultra RNA Library Prep Kit for Illumina (New England Biolabs). cDNA libraries were purified with AMPure XP beads (Beckman Coulter, Brea, CA) and quantified using a Qubit 4 Fluorometer (Thermo Fisher Scientific). Equimolar amounts of barcoded libraries were pooled and sequenced on a NextSeq 500 Sequencing System (Illumina, San Diego, CA) with a 1×75 configuration.

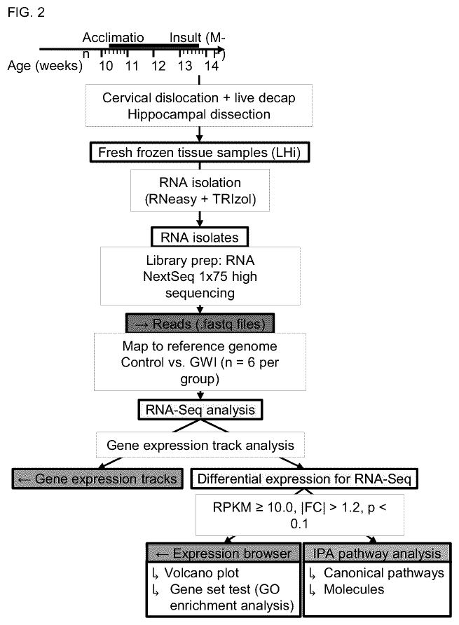

RNA-Seq analysis. RNA-Seq reads were imported into CLC Genomics Workbench (version 20.0.3, QIAGEN) for analysis using a modified version of the workflow for RNA-Seq analysis with export to IPA (FIG. 2). The reads were batch processed and mapped to the Mus musculus reference genome. Control vs. PB+CPF+DEET samples were quantified using the Differential Analysis for RNA-Seq tool. Differentially expressed genes were considered significant if they met the following criteria: mean reads per kilobase of transcript per million mapped reads (RPKM)>10.0, fold change in either direction ≥1.2, and p<0.1. Gene ontology (GO) categories were assigned and analyzed for significance for biological processes, molecular functions, and cellular components using the Gene Set Test tool. GO categories were considered significant if fold change in either direction ≥1.2 and p<0.05. Significant genes were exported to IPA.

Functional analyses were generated using Ingenuity Pathway Analysis (IPA) (QIAGEN). Core analysis was performed on dataset based on RPKM values for genes that met criteria for significance, which generated lists of significant canonical pathways, upstream regulators, associated diseases and functions, and differentially expressed genes. Canonical pathways were based on significant differentially expressed genes, and a pathway itself was considered significant if p<0.05.

Statistics. The statistical analyses for behavior were conducted using GraphPad Prism for macOS (version 9.0.0). Mean values for behavioral analyses are depicted±standard error of the mean (SEM). Data for open field and Y-maze tasks were analyzed using an unpaired t-test with Welch's correction, and statistical significance was considered when p<0.05. Entries into each arm during the Y-maze task were analyzed using multiple unpaired t-tests followed by FDR control with the two-stage step-up method of Benjamini, Krieger, and Yekutieli as recommended by GraphPad. Significant fold changes in RNA expression were analyzed by CLC Genomics Workbench using Differential Expression for RNA-Seq as part of the workflow as detailed in FIG. 2.

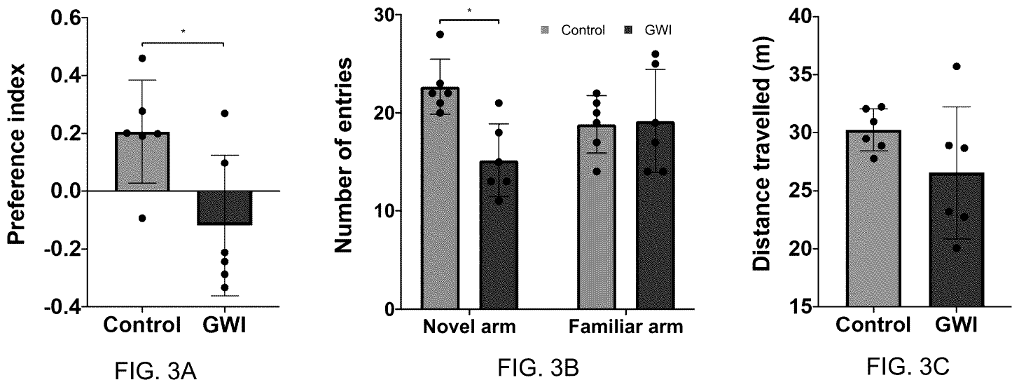

Results. Effects of Gulf War toxicant exposure on hippocampal-dependent spatial memory in Y-maze task. To assess effects of the exposure on hippocampal-dependent spatial memory, mice underwent a Y-maze task (n=6/group). Time spent in each arm, number of entries into each arm, and distance travelled were recorded. Preference for the novel arm was significantly lower by 157% in mice exposed to PB+CPF+DEET compared to controls, p=0.027 (FIG. 3A). The number of entries into the novel arm was also significantly reduced by 33% compared to control mice, p=0.003 (FIG. 3B). Distance travelled during the test stage was 12% lower in toxicant-exposed mice compared to controls and therefore did not significantly differ between conditions, p=0.182 (FIG. 3C).

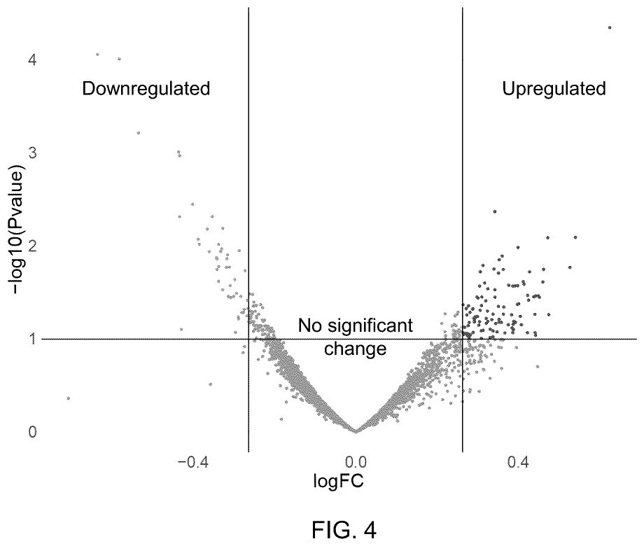

Gene dysregulation after acute exposure to Gulf War toxicants. In the hippocampus, 158 dysregulated genes were identified with the aid of RNA-Seq analysis which met criteria for differential expression in response to Gulf War toxicant exposure (FIG. 4, Tables 1 and 2). A gene set test (GO enrichment analysis) in CLC Genomics Workbench showed significantly affected gene ontology categories. Of these categories, 47 were related to biological processes (Table 4A), 138 were related to molecular functions (Table 4B), and 120 were related to cellular components (Table 4C). Pathway analysis in IPA showed 45 significantly affected canonical pathways (Table 3).

TABLE 1
Downregulated genes after exposure to Gulf War insult.

| Symbol | Entrez Gene Name | RPKM | FC | P-value |
|---|---|---|---|---|
| Arc | activity regulated cytoskeleton | 33.6 | −1.553 | 8.72E−05 |
| | associated protein | | | |
| Egr1 | early growth response 1 | 23.3 | −1.497 | 9.77E−05 |
| Nr4a1 | nuclear receptor subfamily 4 group A | 16.5 | −1.449 | 0.000608 |
| | member 1 | | | |
| Apod | apolipoprotein D | 32.5 | −1.353 | 0.000973 |
| Hba-a2 | hemoglobin alpha, adult chain 2 | 60.7 | −1.350 | 0.00485 |
| Tmem88b | transmembrane protein 88B | 16.5 | −1.350 | 0.00107 |
| Wfs1 | wolframin ER transmembrane | 33.2 | −1.321 | 0.00357 |
| | glycoprotein | | | |
| Junb | JunB proto-oncogene, AP-1 | 36.6 | −1.308 | 0.00847 |
| | transcription factor subunit | | | |
| Fam163 | family with sequence similarity 163 | 52.3 | −1.306 | 0.00959 |
| b | member B | | | |
| Mog | myelin oligodendrocyte glycoprotein | 22.7 | −1.288 | 0.00659 |
| Mbp | myelin basic protein | 281.4 | −1.284 | 0.0115 |
| Bcas1 | breast carcinoma amplified sequence 1 | 41.5 | −1.277 | 0.00483 |
| Cd9 | CD9 molecule | 29.2 | −1.268 | 0.0134 |
| Gsn | gelsolin | 15.9 | −1.267 | 0.0138 |
| Pllp | plasmolipin | 21.3 | −1.267 | 0.018 |
| Mag | myelin associated glycoprotein | 50.6 | −1.263 | 0.00963 |
| Nutf2- | nuclear transport factor 2, pseudogene 1 | 19.1 | −1.263 | 0.0167 |
| ps1 | | | | |
| Pcp4l1 | Purkinje cell protein 4-like 1 | 26.7 | −1.263 | 0.0239 |
| H2-D1 | histocompatibility 2, D region locus 1 | 11.8 | −1.259 | 0.0115 |
| Trf | transferrin | 62.8 | −1.257 | 0.0107 |
| Rpl10- | ribosomal protein L10, pseudogene 3 | 75.6 | −1.255 | 0.0296 |
| ps3 | | | | |
| Plekhb1 | pleckstrin homology domain containing | 65.3 | −1.254 | 0.00647 |
| | B1 | | | |
| Srebf1 | sterol regulatory element binding | 11.2 | −1.247 | 0.017 |
| | transcription factor 1 | | | |
| Cnp | 2′,3′-cyclic nucleotide 3′ | 105.7 | −1.246 | 0.0137 |
| | phosphodiesterase | | | |
| Septin4 | septin 4 | 27.6 | −1.244 | 0.0125 |
| Slco1c1 | solute carrier organic anion transporter | 11.2 | −1.243 | 0.0349 |
| | family member 1C1 | | | |
| Pltp | phospholipid transfer protein | 21.6 | −1.242 | 0.0352 |
| Cldn11 | claudin 11 | 73.5 | −1.240 | 0.0169 |
| Fa2h | fatty acid 2-hydroxylase | 11.0 | −1.239 | 0.0267 |
| Rhog | ras homolog family member G | 12.3 | −1.238 | 0.0359 |
| Prr18 | proline rich 18 | 17.0 | −1.229 | 0.0231 |
| Egr4 | early growth response 4 | 16.8 | −1.228 | 0.0849 |
| mt-Atp8 | ATP synthase F0 subunit 8 | 7509.7 | −1.225 | 0.0323 |
| C1ql2 | complement C1q like 2 | 40.4 | −1.222 | 0.0578 |
| Nfkbia | NFKB inhibitor alpha | 12.4 | −1.221 | 0.0608 |
| Igfbp5 | insulin like growth factor binding | 23.2 | −1.219 | 0.0112 |
| | protein 5 | | | |
| B2m | beta-2-microglobulin | 57.4 | −1.217 | 0.0348 |
| Hbb-bs | hemoglobin subunit beta | 45.2 | −1.215 | 0.0521 |
| S100a16 | S100 calcium binding protein A16 | 20.3 | −1.211 | 0.0518 |
| mt-Atp6 | ATP synthase F0 subunit 6 | 8936.0 | −1.210 | 0.0481 |
| Slc6a6 | solute carrier family 6 member 6 | 15.6 | −1.208 | 0.0185 |
| Ddit4 | DNA damage inducible transcript 4 | 38.8 | −1.204 | 0.0563 |
| Anxa5 | annexin A5 | 15.5 | −1.203 | 0.0383 |
| S100a1 | S100 calcium binding protein A1 | 35.3 | −1.202 | 0.0469 |
| Chrm3 | cholinergic receptor muscarinic 3 | 10.5 | −1.200 | 0.0652 |

BOLD, negative fold changes indicate downregulation.

TABLE 2
Upregulated genes after exposure to Gulf War insult.

| Symbol | Entrez Gene Name | RPKM | FC | P-value |
|---|---|---|---|---|
| Lars2 | leucyl-tRNA synthetase 2, mitochondrial | 744.328 | 1.542 | 4.48E−05 |
| Gdf1 | growth differentiation factor 1 | 11.348 | 1.454 | 0.0081 |
| Cdr1 | cerebellar degeneration related antigen 1 | 23.178 | 1.441 | 0.017 |
| Fam126b | family with sequence similarity 126 member B | 12.032 | 1.390 | 0.0548 |
| Pak3 | p21 (RAC1) activated kinase 3 | 14.277 | 1.387 | 0.00816 |
| Igip | IgA inducing protein | 31.362 | 1.377 | 0.0179 |
| Pgm2l1 | phosphoglucomutase 2 like 1 | 45.308 | 1.376 | 0.0244 |
| Smc3 | structural maintenance of chromosomes 3 | 11.216 | 1.367 | 0.0357 |
| Dgkb | diacylglycerol kinase beta | 20.952 | 1.365 | 0.0343 |
| Atrx | ATRX chromatin remodeler | 10.147 | 1.359 | 0.0912 |
| Ppp4r2 | protein phosphatase 4 regulatory subunit 2 | 14.623 | 1.359 | 0.0345 |
| Ankrd12 | ankyrin repeat domain 12 | 10.712 | 1.357 | 0.0856 |
| Hspa4l | heat shock protein family A (Hsp70) member 4 | 12.354 | 1.357 | 0.0544 |
| | like | | | |
| Ppig | peptidylprolyl isomerase G | 11.563 | 1.354 | 0.0353 |
| Rabep1 | rabaptin, RAB GTPase binding effector protein 1 | 16.266 | 1.345 | 0.019 |
| Dnajb4 | DnaJ heat shock protein family (Hsp40) member | 11.871 | 1.343 | 0.0384 |
| | B4 | | | |
| Pcmtd1 | protein-L-isoaspartate (D-aspartate) O- | 16.76 | 1.340 | 0.0856 |
| | methyltransferase domain containing 1 | | | |
| Reps2 | RALBP1 associated Eps domain containing 2 | 21.405 | 1.340 | 0.034 |
| Ube2q2 | ubiquitin conjugating enzyme E2 Q2 | 13.79 | 1.332 | 0.0259 |
| Rab3c | RAB3C, member RAS oncogene family | 44.551 | 1.330 | 0.0242 |
| Acbd5 | acyl-CoA binding domain containing 5 | 11.406 | 1.326 | 0.0703 |
| Fmr1 | FMRP translational regulator 1 | 11.502 | 1.324 | 0.0571 |
| Tax1bp1 | Tax1 binding protein 1 | 22.074 | 1.320 | 0.0419 |
| Nus1 | NUS1 dehydrodolichyl diphosphate synthase | 16.346 | 1.319 | 0.0104 |
| | subunit | | | |
| Hsp90aa1 | heat shock protein 90 alpha family class A | 213.841 | 1.318 | 0.0268 |
| | member 1 | | | |
| Gmfb | glia maturation factor beta | 35.439 | 1.317 | 0.0425 |
| Gpbp1 | GC-rich promoter binding protein 1 | 17.787 | 1.313 | 0.0663 |
| Naa50 | N(alpha)-acetyltransferase 50, NatE catalytic | 16.618 | 1.312 | 0.0269 |
| | subunit | | | |
| Gabra2 | gamma-aminobutyric acid type A receptor | 36.465 | 1.309 | 0.0647 |
| | alpha2 subunit | | | |
| Fxr1 | FMR1 autosomal homolog 1 | 11.598 | 1.308 | 0.0792 |
| Kpna3 | karyopherin subunit alpha 3 | 16.133 | 1.308 | 0.0695 |
| Ipo7 | importin 7 | 17.7 | 1.307 | 0.0853 |
| Mphosph8 | M-phase phosphoprotein 8 | 15.901 | 1.307 | 0.027 |
| Kif5b | kinesin family member 5B | 33.739 | 1.305 | 0.0762 |
| Psd3 | pleckstrin and Sec7 domain containing 3 | 26.285 | 1.304 | 0.0549 |
| Pde1a | phosphodiesterase 1A | 26.28 | 1.298 | 0.0265 |
| Mob4 | MOB family member 4, phocein | 14.856 | 1.297 | 0.0703 |
| Uba3 | ubiquitin like modifier activating enzyme 3 | 13.952 | 1.289 | 0.086 |
| Slc8a1 | solute carrier family 8 member A1 | 11.848 | 1.287 | 0.0454 |
| Ankrd13c | ankyrin repeat domain 13C | 19.116 | 1.286 | 0.0179 |
| Pten | phosphatase and tensin homolog | 20.14 | 1.286 | 0.0542 |
| Eif3a | eukaryotic translation initiation factor 3 subunit | 27.913 | 1.284 | 0.0128 |
| | A | | | |
| Gabrb1 | gamma-aminobutyric acid type A receptor beta1 | 13.845 | 1.282 | 0.0663 |
| | subunit | | | |
| Ogfrl1 | opioid growth factor receptor like 1 | 36.947 | 1.277 | 0.0141 |
| Selenot | selenoprotein T | 44.851 | 1.277 | 0.0616 |
| Eif5 | eukaryotic translation initiation factor 5 | 34.859 | 1.276 | 0.0662 |
| Htatsf1 | HIV-1 Tat specific factor 1 | 18.843 | 1.275 | 0.0447 |
| Top1 | DNA topoisomerase I | 22.617 | 1.275 | 0.0196 |
| Slc25a46 | solute carrier family 25 member 46 | 11.765 | 1.272 | 0.084 |
| Nrxn1 | neurexin 1 | 30.941 | 1.269 | 0.0682 |
| Gad2 | glutamate decarboxylase 2 | 17.309 | 1.268 | 0.0356 |
| Fgfr1op2 | FGFR1 oncogene partner 2 | 25.756 | 1.267 | 0.0296 |
| Hspa5 | heat shock protein family A (Hsp70) member 5 | 46.918 | 1.267 | 0.00428 |
| Zc3h15 | zinc finger CCCH-type containing 15 | 34.721 | 1.266 | 0.0177 |
| Armcx3 | armadillo repeat containing X-linked 3 | 22 | 1.264 | 0.0541 |
| Hnrnpa3 | heterogeneous nuclear ribonucleoprotein A3 | 29.364 | 1.263 | 0.0946 |
| Senp6 | SUMO specific peptidase 6 | 10.207 | 1.263 | 0.0819 |
| Fbxo11 | F-box protein 11 | 23.116 | 1.261 | 0.062 |
| Cert1 | ceramide transporter 1 | 11.829 | 1.257 | 0.0968 |
| Oxr1 | oxidation resistance 1 | 23.222 | 1.257 | 0.0785 |
| Impact | impact RWD domain protein | 38.33 | 1.252 | 0.0648 |
| Psip1 | PC4 and SFRS1 interacting protein 1 | 32.895 | 1.252 | 0.0289 |
| Slmap | sarcolemma associated protein | 13.2 | 1.252 | 0.0502 |
| Fgf12 | fibroblast growth factor 12 | 10.635 | 1.249 | 0.0679 |
| Sucla2 | succinate-CoA ligase ADP-forming beta subunit | 33.008 | 1.249 | 0.0601 |
| Dld | dihydrolipoamide dehydrogenase | 28.74 | 1.248 | 0.0389 |
| Negr1 | neuronal growth regulator 1 | 18.551 | 1.246 | 0.0251 |
| Acsl4 | acyl-CoA synthetase long chain family member | 13.462 | 1.242 | 0.0806 |
| | 4 | | | |
| Dnaja1 | DnaJ heat shock protein family (Hsp40) member | 37.903 | 1.242 | 0.0162 |
| | A1 | | | |
| Pnrc2 | proline rich nuclear receptor coactivator 2 | 13.435 | 1.242 | 0.0808 |
| Eif5b | eukaryotic translation initiation factor 5B | 11.453 | 1.240 | 0.0354 |
| Mib1 | mindbomb E3 ubiquitin protein ligase 1 | 15.309 | 1.239 | 0.0985 |
| Plcb1 | phospholipase C beta 1 | 19.494 | 1.239 | 0.0438 |
| Map9 | microtubule associated protein 9 | 15.383 | 1.238 | 0.0815 |
| Jakmip2 | janus kinase and microtubule interacting protein | 11.357 | 1.236 | 0.0491 |
| | 2 | | | |
| Pura | purine rich element binding protein A | 19.084 | 1.236 | 0.019 |
| Hsp90b1 | heat shock protein 90 beta family member 1 | 65.468 | 1.235 | 0.027 |
| Ncl | nucleolin | 18.087 | 1.235 | 0.0652 |
| Neto1 | neuropilin and tolloid like 1 | 16.105 | 1.233 | 0.0711 |
| Gda | guanine deaminase | 30.573 | 1.232 | 0.0364 |
| Cnr1 | cannabinoid receptor 1 | 25.574 | 1.231 | 0.0575 |
| Bhlhb9 | basic helix-loop-helix family member b9 | 16.07 | 1.229 | 0.0355 |
| Ythdc1 | YTH domain containing 1 | 13.542 | 1.228 | 0.0578 |
| Golga4 | golgin A4 | 10.017 | 1.226 | 0.0576 |
| Cir1 | corepressor interacting with RBPJ, 1 | 10.903 | 1.224 | 0.0657 |
| Mzt1 | mitotic spindle organizing protein 1 | 27.367 | 1.224 | 0.0631 |
| Rnf6 | ring finger protein 6 | 10.488 | 1.224 | 0.0599 |
| Gdap1 | ganglioside induced differentiation associated | 20.359 | 1.223 | 0.0596 |
| | protein 1 | | | |
| Lpgat1 | lysophosphatidylglycerol acyltransferase 1 | 20.943 | 1.221 | 0.0473 |
| Pin4 | peptidylprolyl cis/trans isomerase, NIMA- | 14.967 | 1.221 | 0.085 |
| | interacting 4 | | | |
| Cpne7 | copine 7 | 93.847 | 1.220 | 0.0478 |
| Ggnbp2 | gametogenetin binding protein 2 | 18.355 | 1.216 | 0.0902 |
| Etv1 | ETS variant transcription factor 1 | 14.066 | 1.215 | 0.0518 |
| Arl5a | ADP ribosylation factor like GTPase 5A | 15.424 | 1.214 | 0.0924 |
| Pafah1b1 | platelet activating factor acetylhydrolase 1b | 59.969 | 1.213 | 0.0964 |
| | regulatory subunit 1 | | | |
| Tafa1 | TAFA chemokine like family member 1 | 11.298 | 1.213 | 0.0663 |
| Srsf3 | serine and arginine rich splicing factor 3 | 27.672 | 1.212 | 0.044 |
| Tceal9 | transcription elongation factor A like 9 | 29.46 | 1.212 | 0.0939 |
| Ccdc47 | coiled-coil domain containing 47 | 14.385 | 1.211 | 0.0885 |
| Tim2 | tripartite motif containing 2 | 46.277 | 1.211 | 0.072 |
| Aff4 | AF4/FMR2 family member 4 | 15.746 | 1.210 | 0.093 |
| C5orf24 | chromosome 5 open reading frame 24 | 18.903 | 1.210 | 0.0917 |
| Msantd4 | Myb/SANT DNA binding domain containing 4 | 14.084 | 1.208 | 0.0552 |
| | with coiled-coils | | | |
| Rab39b | RAB39B, member RAS oncogene family | 10.444 | 1.208 | 0.087 |
| Vxn | vexin | 56.093 | 1.207 | 0.0472 |
| Tmem33 | transmembrane protein 33 | 10.859 | 1.205 | 0.0876 |
| Slk | STE20 like kinase | 10.826 | 1.204 | 0.0747 |
| Hdgfl3 | HDGF like 3 | 11.325 | 1.202 | 0.0891 |
| Dynlt3 | dynein light chain Tctex-type 3 | 39.421 | 1.201 | 0.0859 |
| Dyrk2 | dual specificity tyrosine phosphorylation | 10.698 | 1.200 | 0.0429 |
| | regulated kinase 2 | | | |

BOLD, positive folder changes indicate upregulation.

TABLE 3
Significantly affected canonical pathways after Gulf War insult.

| | -log(p- | | |
|---|---|---|---|
| Ingenuity Canonical Pathways | value) | Ratio | Molecules |
| Protein Ubiquitination Pathway | 4.08 | 0.033 | B2m, Dnajal, Dnajb4, Hba-a2, |
| | | | Hsp90aa1, Hsp90b1, Hspa41, Hspa5, |
| | | | Ube2q2 |
| Aldosterone Signaling in Epithelial | 4.07 | 0.0443 | Dnaja1, Dnajb4, Hsp90aal, Hsp90b1, |
| Cells | | | Hspa41, Hspa5, Plcb1 |
| Hypoxia Signaling in the | 3.9 | 0.0676 | Hsp90aa1, Hsp90b1, Nfkbia, Pten, |
| Cardiovascular System | | | Ube2q2 |
| Mitotic Roles of Polo-Like Kinase | 3.03 | 0.0606 | Hsp90aa1, Hsp90b1, Slk, Smc3 |
| Prostate Cancer Signaling | 2.51 | 0.044 | Hsp90aa1, Hsp90b1, Nfkbia, Pten |
| Unfolded protein response | 2.23 | 0.0536 | Hsp90b1, Hspa5, Srebf1 |
| Role of PKR in Interferon Induction | 2.13 | 0.0342 | Hsp90aa1, Hsp90b1, Hspa5, Nfkbia |
| and Antiviral Response | | | |
| Endoplasmic Reticulum Stress | 2.08 | 0.0952 | Hsp90b1, Hspa5 |
| Pathway | | | |
| LXR/RXR Activation | 2.08 | 0.0331 | Apod, Pltp, Srebf1, Trf |
| FXR/RXR Activation | 2.02 | 0.0317 | Apod, Pltp, Srebf1, Trf |
| TCA Cycle II (Eukaryotic) | 1.97 | 0.0833 | Dld, Sucla2 |
| Glutamate Dependent Acid | 1.88 | 0.5 | Gad2 |
| Resistance | | | |
| EIF2 Signaling | 1.79 | 0.0223 | Eif3a, Eif5, Eif5b, Hspa5, Srebf1 |
| Gαq Signaling | 1.69 | 0.0253 | Chrm3, Nfkbia, Plcb1, Rhog |
| Cytotoxic T Lymphocyte-mediated | 1.68 | 0.0588 | B2m, Hba-a2 |
| Apoptosis of Target Cells | | | |
| eNOS Signaling | 1.68 | 0.0252 | Chrm3, Hsp90aa1, Hsp90b1, Hspa5 |
| OX40 Signaling Pathway | 1.67 | 0.0333 | B2m, Hba-a2, Nfkbia |
| Regulation of Actin-based Motility | 1.62 | 0.0319 | Gsn, Pak3, Rhog |
| by Rho | | | |
| CXCR4 Signaling | 1.61 | 0.024 | Egr1, Pak3, Plcb1, Rhog |
| GABA Receptor Signaling | 1.61 | 0.0316 | Gabra2, Gabrb1, Gad2 |
| Neuregulin Signaling | 1.6 | 0.0312 | Hsp90aa1, Hsp90b1, Pten |
| Branched-chain α-keto acid | 1.59 | 0.25 | Dld |
| Dehydrogenase Complex | | | |
| Antigen Presentation Pathway | 1.57 | 0.0513 | B2m, Hba-a2 |
| Nitric Oxide Signaling in the | 1.56 | 0.0303 | Hsp90aa1, Hsp90b1, Pde1A |
| Cardiovascular System | | | |
| PI3K/AKT Signaling | 1.55 | 0.0229 | Hsp90aal, Hsp90b1, Nfkbia, Pten |
| Sumoylation Pathway | 1.52 | 0.0291 | Nfkbia, Rhog, Senp6 |
| PPAR Signaling | 1.51 | 0.0288 | Hsp90aa1, Hsp90b1, Nfkbia |
| 2-ketoglutarate Dehydrogenase | 1.49 | 0.2 | Dld |
| Complex | | | |
| 2-oxobutanoate Degradation I | 1.49 | 0.2 | Dld |
| Glutamate Degradation III (via 4- | 1.49 | 0.2 | Gad2 |
| aminobutyrate) | | | |
| BAG2 Signaling Pathway | 1.49 | 0.0465 | Hsp90aal, Hspa5 |
| Dendritic Cell Maturation | 1.49 | 0.0219 | B2m, Hba-a2, Nfkbia, Plcb1 |
| PD-1, PD-LI cancer | 1.49 | 0.0283 | B2m, Hba-a2, Pten |
| immunotherapy pathway | | | |
| G-Protein Coupled Receptor | 1.47 | 0.0184 | Chrm3, Cnr1, Nfkbia, Pde1A, Plcb1 |
| Signaling | | | |
| Antioxidant Action of Vitamin C | 1.45 | 0.0275 | Nfkbia, Plcb1, Selenot |
| PPARα/RXRα Activation | 1.44 | 0.0211 | Hsp90aa1, Hsp90b1, Nfkbia, Plcb1 |
| iCOS-iCOSL Signaling in T Helper | 1.44 | 0.027 | Hba-a2, Nfkbia, Pten |
| Cells | | | |
| Type I Diabetes Mellitus Signaling | 1.44 | 0.027 | Gad2, Hba-a2, Nfkbia |
| Glycine Cleavage Complex | 1.41 | 0.167 | Dld |
| Natural Killer Cell Signaling | 1.39 | 0.0203 | B2m, Hba-a2, Hspa5, Pak3 |
| Role of Tissue Factor in Cancer | 1.38 | 0.0256 | Egr1, Plcb1, Pten |
| TNFR1 Signaling | 1.37 | 0.04 | Nfkbia, Pak3 |
| Thioredoxin Pathway | 1.35 | 0.143 | Selenot |
| Acetyl-CoA Biosynthesis I | 1.35 | 0.143 | Dld |
| (Pyruvate Dehydrogenase | | | |
| Complex) | | | |
| Neuroinflammation Signaling | 1.32 | 0.0167 | B2m, Gabra2, Gabrb1, Gad2, Hba-a2 |
| Pathway | | | |

BOLD, negative fold changes indicate downregulation.
Underline, positive fold changes indicate upregulation.

| | | Detected | DE | DE Genes | |
|---|---|---|---|---|---|
| GO term | Description | Genes | Genes | (Names) | P-values |
| 0110077 | vesicle-mediated intercellular | 1 | 1 | Arc | 2.89E−4 |
| | transport | | | | |
| 0006429 | leucyl-tRNA aminoacylation | 2 | 1 | Lars2 | 5.78E−4 |
| 0050767 | regulation of neurogenesis | 934 | 3 | Arc, Gh, | 1.45E−3 |
| | | | | Opalin | |
| 0006518 | peptide metabolic process | 262 | 2 | Hmgn5, | 2.22E−3 |
| | | | | Lars2 | |
| 0090031 | positive regulation of steroid | 9 | 1 | Gh | 2.60E−3 |
| | hormone biosynthetic process | | | | |
| 2000969 | positive regulation of alpha- | 9 | 1 | Arc | 2.60E−3 |
| | amino-3-hydroxy-5-methyl-4- | | | | |
| | isoxazole propionate selective | | | | |
| | glutamate receptor activity | | | | |
| 0032094 | response to food | 12 | 1 | Gh | 3.47E−3 |
| 0043603 | cellular amide metabolic process | 392 | 2 | Hmgn5, | 4.90E−3 |
| | | | | Lars2 | |
| 1900452 | regulation of long-term synaptic | 17 | 1 | Arc | 4.91E−3 |
| | depression | | | | |
| 0007405 | neuroblast proliferation | 19 | 1 | Gh | 5.48E−3 |
| 0099149 | regulation of postsynaptic | 23 | 1 | Arc | 6.64E−3 |
| | neurotransmitter receptor | | | | |
| | internalization | | | | |
| 0032543 | mitochondrial translation | 31 | 1 | Lars2 | 8.94E−3 |
| 0007616 | long-term memory | 39 | 1 | Arc | 0.0112 |
| 0007492 | endoderm development | 40 | 1 | Arc | 0.0115 |
| 0040018 | positive regulation of | 42 | 1 | Gh | 0.0121 |
| | multicellular organism growth | | | | |
| 0072089 | stem cell proliferation | 45 | 1 | Gh | 0.0129 |
| 0048286 | lung alveolus development | 49 | 1 | Gh | 0.0141 |
| 0048713 | regulation of oligodendrocyte | 49 | 1 | Opalin | 0.0141 |
| | differentiation | | | | |
| 0010828 | positive regulation of glucose | 50 | 1 | Gh | 0.0144 |
| | transport | | | | |
| 0051028 | mRNA transport | 52 | 1 | Arc | 0.0149 |
| 1900271 | regulation of long-term synaptic | 54 | 1 | Arc | 0.0155 |
| | potentiation | | | | |
| 0006749 | glutathione metabolic process | 55 | 1 | Hmgn5 | 0.0158 |
| 0061001 | regulation of dendritic spine | 56 | 1 | Arc | 0.0161 |
| | morphogenesis | | | | |
| 0099601 | regulation of neurotransmitter | 60 | 1 | Arc | 0.0172 |
| | receptor activity | | | | |
| 0061351 | neural precursor cell proliferation | 63 | 1 | Gh | 0.0181 |
| 0048168 | regulation of neuronal synaptic | 69 | 1 | Arc | 0.0198 |
| | plasticity | | | | |
| 0045685 | regulation of glial cell | 86 | 1 | Opalin | 0.0246 |
| | differentiation | | | | |
| 0032869 | cellular response to insulin | 87 | 1 | Gh | 0.0249 |
| | stimulus | | | | |
| 0046889 | positive regulation of lipid | 94 | 1 | Gh | 0.0269 |
| | biosynthetic process | | | | |
| 0060998 | regulation of dendritic spine | 97 | 1 | Arc | 0.0277 |
| | development | | | | |
| 0032414 | positive regulation of ion | 114 | 1 | Arc | 0.0325 |
| | transmembrane transporter | | | | |
| | activity | | | | |
| 0051260 | protein homooligomerization | 116 | 1 | Arc | 0.0331 |
| 0071375 | cellular response to peptide | 119 | 1 | Gh | 0.0340 |
| | hormone stimulus | | | | |
| 0006575 | cellular modified amino acid | 138 | 1 | Hmgn5 | 0.0393 |
| | metabolic process | | | | |
| 0014013 | regulation of gliogenesis | 148 | 1 | Opalin | 0.0421 |
| 1901564 | organonitrogen compound | 1208 | 2 | Hmgn5, | 0.0423 |
| | metabolic process | | | Lars2 | |
| 0010469 | regulation of receptor activity | 156 | 1 | Arc | 0.0443 |
| 0009952 | anterior/posterior pattern | 170 | 1 | Arc | 0.0482 |
| | specification | | | | |
| 0043604 | amide biosynthetic process | 214 | 1 | Lars2 | 0.0604 |
| 0043933 | macromolecular complex subunit | 1504 | 2 | Arc, | 0.0633 |
| | organization | | | Hmgn5 | |
| 1901215 | negative regulation of neuron | 244 | 1 | Gh | 0.0686 |
| | death | | | | |
| 0032412 | regulation of ion transmembrane | 249 | 1 | Arc | 0.0700 |
| | transporter activity | | | | |
| 0006790 | sulfur compound metabolic | 271 | 1 | Hmgn5 | 0.0760 |
| | process | | | | |
| 0009416 | response to light stimulus | 288 | 1 | Gh | 0.0806 |
| 0050890 | cognition | 325 | 1 | Arc | 0.0905 |
| 0010769 | regulation of cell morphogenesis | 344 | 1 | Arc | 0.0956 |
| | involved in differentiation | | | | |
| 0007005 | mitochondrion organization | 345 | 1 | Lars2 | 0.0959 |
| 0071417 | cellular response to | 347 | 1 | Gh | 0.0964 |
| | organonitrogen compound | | | | |

| | | Detected | DE | DE Genes | |
|---|---|---|---|---|---|
| GO term | Description | Genes | Genes | (Names) | P-values |
| 0033592 | RNA strand annealing | 3 | 2 | Fmr1, Fxr1 | 2.18E−04 |
| | activity | | | | |
| 0097100 | supercoiled DNA binding | 3 | 2 | Psip1, Top1 | 2.18E−04 |
| 0070840 | dynein complex binding | 21 | 3 | Fmr1, Pafah1b1, | 7.32E−04 |
| | | | | Smc3 | |
| 0051082 | unfolded protein binding | 61 | 4 | Dnajb4, | 1.85E−03 |
| | | | | Hsp90aa1, | |
| | | | | Hsp90b1, Hspa5 | |
| 0002151 | G-quadruplex RNA | 9 | 2 | Fmr1, Fxr1 | 2.52E−03 |
| | binding | | | | |
| 0062061 | TAP complex binding | 9 | 2 | H2-D1, H2-K1 | 2.52E−03 |
| 0031720 | haptoglobin binding | 9 | 2 | Hba-a2, Hbb-bs | 2.52E−03 |
| 0019911 | structural constituent of | 10 | 2 | Mbp, Pllp | 3.14E−03 |
| | myelin sheath | | | | |
| 0030881 | beta-2-microglobulin | 11 | 2 | H2-D1, H2-K1 | 3.81E−03 |
| | binding | | | | |
| 0042610 | CD8 receptor binding | 11 | 2 | H2-D1, H2-K1 | 3.81E−03 |
| 0046977 | TAP binding | 11 | 2 | H2-D1, H2-K1 | 3.81E−03 |
| 0003743 | translation initiation | 38 | 3 | Eif3a, Eif5, Eif5b | 4.18E−03 |
| | factor activity | | | | |
| 1990825 | sequence-specific mRNA | 13 | 2 | Fmr1, Srsf3 | 5.35E−03 |
| | binding | | | | |
| 0022851 | GABA-gated chloride | 13 | 2 | Gabra2, Gabrb1 | 5.35E−03 |
| | ion channel activity | | | | |
| 0097001 | ceramide binding | 14 | 2 | Mag, Pltp | 6.20E−03 |
| 0042608 | T cell receptor binding | 15 | 2 | H2-D1, H2-K1 | 7.12E−03 |
| 0004113 | 2′,3′-cyclic-nucleotide | 1 | 1 | Cnp | 8.57E−03 |
| | 3′-phosphodiesterase | | | | |
| | activity | | | | |
| 0004148 | dihydrolipoyl | 1 | 1 | Dld | 8.57E−03 |
| | dehydrogenase activity | | | | |
| 0043544 | lipoamide binding | 1 | 1 | Dld | 8.57E−03 |
| 0080132 | fatty acid alpha- | 1 | 1 | Fa2h | 8.57E−03 |
| | hydroxylase activity | | | | |
| 0008892 | guanine deaminase | 1 | 1 | Gda | 8.57E−03 |
| | activity | | | | |
| 0052858 | peptidyl-lysine | 1 | 1 | Naa50 | 8.57E−03 |
| | acetyltransferase activity | | | | |
| 1990631 | ErbB-4 class receptor | 1 | 1 | Ncl | 8.57E−03 |
| | binding | | | | |
| 0047933 | glucose-1,6-bisphosphate | 1 | 1 | Pgm2l1 | 8.57E−03 |
| | synthase activity | | | | |
| 0140339 | phosphatidylglycerol | 1 | 1 | Pltp | 8.57E−03 |
| | transfer activity | | | | |
| 0140340 | cerebroside transfer | 1 | 1 | Pltp | 8.57E−03 |
| | activity | | | | |
| 0140337 | diacylglyceride transfer | 1 | 1 | Pltp | 8.57E−03 |
| | activity | | | | |
| 0140338 | sphingomyelin transfer | 1 | 1 | Pltp | 8.57E−03 |
| | activity | | | | |
| 0051717 | inositol-1,3,4,5- | 1 | 1 | Pten | 8.57E−03 |
| | tetrakisphosphate 3- | | | | |
| | phosphatase activity | | | | |
| 0051800 | phosphatidylinositol-3,4- | 1 | 1 | Pten | 8.57E−03 |
| | bisphosphate 3- | | | | |
| | phosphatase activity | | | | |
| 0001761 | beta-alanine | 1 | 1 | Slc6a6 | 8.57E−03 |
| | transmembrane | | | | |
| | transporter activity | | | | |
| 0005369 | taurine:sodium symporter | 1 | 1 | Slc6a6 | 8.57E−03 |
| | activity | | | | |
| 0004890 | GABA-A receptor | 18 | 2 | Gabra2, Gabrb1 | 0.010198 |
| | activity | | | | |
| 0019825 | oxygen binding | 18 | 2 | Hba-a2, Hbb-bs | 0.010198 |
| 0031489 | myosin V binding | 20 | 2 | Rab39b, Rab3c | 0.012524 |
| 0043022 | ribosome binding | 57 | 3 | Fmr1, Hspa5, | 0.01287 |
| | | | | Impact | |
| 0008139 | nuclear localization | 21 | 2 | Kpna3, Nfkbia | 0.013765 |
| | sequence binding | | | | |
| 0005104 | fibroblast growth factor | 22 | 2 | Fgf12, Nrxn1 | 0.015057 |
| | receptor binding | | | | |
| 0001671 | ATPase activator activity | 23 | 2 | Dnaja1, Dnajb4 | 0.0164 |
| 0004351 | glutamate decarboxylase | 2 | 1 | Gad2 | 0.017065 |
| | activity | | | | |
| 0031722 | hemoglobin beta binding | 2 | 1 | Hbb-bs | 0.017065 |
| 0002135 | CTP binding | 2 | 1 | Hsp90aa1 | 0.017065 |
| 0099609 | microtubule lateral | 2 | 1 | Kif5b | 0.017065 |
| | binding | | | | |
| 0004823 | leucine-tRNA ligase | 2 | 1 | Lars2 | 0.017065 |
| | activity | | | | |
| 0045547 | dehydrodolichyl | 2 | 1 | Nus1 | 0.017065 |
| | diphosphate synthase | | | | |
| | activity | | | | |
| 0120019 | phosphatidylcholine | 2 | 1 | Pltp | 0.017065 |
| | transfer activity | | | | |
| 0030977 | taurine binding | 2 | 1 | Slc6a6 | 0.017065 |
| 0086038 | calcium:sodium | 2 | 1 | Slc8a1 | 0.017065 |
| | antiporter activity | | | | |
| | involved in regulation of | | | | |
| | cardiac muscle cell | | | | |
| | membrane potential | | | | |
| 0099580 | ion antiporter activity | 2 | 1 | Slc8a1 | 0.017065 |
| | involved in regulation of | | | | |
| | postsynaptic membrane | | | | |
| | potential | | | | |
| 0032810 | sterol response element | 2 | 1 | Srebf1 | 0.017065 |
| | binding | | | | |
| 0004775 | succinate-CoA ligase | 2 | 1 | Sucla2 | 0.017065 |
| | (ADP-forming) activity | | | | |
| 0034986 | iron chaperone activity | 2 | 1 | Trf | 0.017065 |
| 0019781 | NEDD8 activating | 2 | 1 | Uba3 | 0.017065 |
| | enzyme activity | | | | |
| 0048027 | mRNA 5′-UTR binding | 24 | 2 | Fmr1, Ncl | 0.017791 |
| 0044183 | protein folding chaperone | 26 | 2 | Hsp90aa1, Hspa5 | 0.020718 |
| 0035064 | methylated histone | 70 | 3 | Atrx, Fmr1, | 0.022223 |
| | binding | | | Mphosph8 | |
| 0048306 | calcium-dependent | 70 | 3 | Nrxn1, S100a1, | 0.022223 |
| | protein binding | | | Wfs1 | |
| 0042605 | peptide antigen binding | 27 | 2 | H2-D1, H2-K1 | 0.022251 |
| 0042165 | neurotransmitter binding | 28 | 2 | Chrm3, Slc6a6 | 0.02383 |
| 0050750 | low-density lipoprotein | 28 | 2 | Dnaja1, Hsp90b1 | 0.02383 |
| | particle receptor binding | | | | |
| 0008081 | phosphoric diester | 72 | 3 | Cnp, Pde1a, | 0.023917 |
| | hydrolase activity | | | Plcb1 | |
| 0004949 | cannabinoid receptor | 3 | 1 | Cnr1 | 0.025489 |
| | activity | | | | |
| 0044729 | hemi-methylated DNA- | 3 | 1 | Egr1 | 0.025489 |
| | binding | | | | |
| 0051033 | RNA transmembrane | 3 | 1 | Hnrnpa3 | 0.025489 |
| | transporter activity | | | | |
| 0017098 | sulfonylurea receptor | 3 | 1 | Hsp90aa1 | 0.025489 |
| | binding | | | | |
| 1905576 | ganglioside GT1b | 3 | 1 | Mag | 0.025489 |
| | binding | | | | |
| 0042134 | rRNA primary transcript | 3 | 1 | Ncl | 0.025489 |
| | binding | | | | |
| 0004719 | protein-L-isoaspartate | 3 | 1 | Pcmtd1 | 0.025489 |
| | (D-aspartate) O- | | | | |
| | methyltransferase | | | | |
| | activity | | | | |
| 0048101 | calcium- and calmodulin- | 3 | 1 | Pde1a | 0.025489 |
| | regulated 3′,5′-cyclic- | | | | |
| | GMP phosphodiesterase | | | | |
| | activity | | | | |
| 0004117 | calmodulin-dependent | 3 | 1 | Pde1a | 0.025489 |
| | cyclic-nucleotide | | | | |
| | phosphodiesterase | | | | |
| | activity | | | | |
| 0003681 | bent DNA binding | 3 | 1 | Pin4 | 0.025489 |
| 0016314 | phosphatidylinositol- | 3 | 1 | Pten | 0.025489 |
| | 3,4,5-trisphosphate 3- | | | | |
| | phosphatase activity | | | | |
| 0070139 | SUMO-specific | 3 | 1 | Senp6 | 0.025489 |
| | endopeptidase activity | | | | |
| 1905060 | calcium:cation antiporter | 3 | 1 | Slc8a1 | 0.025489 |
| | activity involved in | | | | |
| | regulation of | | | | |
| | postsynaptic cytosolic | | | | |
| | calcium ion | | | | |
| | concentration | | | | |
| 0003917 | DNA topoisomerase type | 3 | 1 | Top1 | 0.025489 |
| | I activity | | | | |
| 0071074 | eukaryotic initiation | 4 | 1 | Eif5 | 0.033841 |
| | factor eIF2 binding | | | | |
| 0031721 | hemoglobin alpha | 4 | 1 | Hbb-bs | 0.033841 |
| | binding | | | | |
| 0032564 | dATP binding | 4 | 1 | Hsp90aa1 | 0.033841 |
| 0032551 | pyrimidine | 4 | 1 | Hsp90aa1 | 0.033841 |
| | ribonucleoside binding | | | | |
| 0002134 | UTP binding | 4 | 1 | Hsp90aa1 | 0.033841 |
| 0044547 | DNA topoisomerase | 4 | 1 | Ncl | 0.033841 |
| | binding | | | | |
| 0097109 | neuroligin family protein | 4 | 1 | Nrxn1 | 0.033841 |
| | binding | | | | |
| 0032422 | purine-rich negative | 4 | 1 | Pura | 0.033841 |
| | regulatory element | | | | |
| | binding | | | | |
| 0015349 | thyroid hormone | 4 | 1 | Slco1c1 | 0.033841 |
| | transmembrane | | | | |
| | transporter activity | | | | |
| 0042162 | telomeric DNA binding | 34 | 2 | Ncl, Pura | 0.03421 |
| 0031369 | translation initiation | 35 | 2 | Eif5, Fmr1 | 0.036084 |
| | factor binding | | | | |
| 0060590 | ATPase regulator activity | 37 | 2 | Dnaja1, Dnajb4 | 0.039946 |
| 0070087 | chromo shadow domain | 5 | 1 | Atrx | 0.042122 |
| | binding | | | | |
| 0015616 | DNA translocase activity | 5 | 1 | Atrx | 0.042122 |
| 0055131 | C3HC4-type RING | 5 | 1 | Dnaja1 | 0.042122 |
| | finger domain binding | | | | |
| 0005131 | growth hormone receptor | 5 | 1 | Gh | 0.042122 |
| | binding | | | | |
| 0051022 | Rho GDP-dissociation | 5 | 1 | Hsp90aa1 | 0.042122 |
| | inhibitor binding | | | | |
| 0005105 | type 1 fibroblast growth | 5 | 1 | Nrxn1 | 0.042122 |
| | factor receptor binding | | | | |
| 0036033 | mediator complex | 5 | 1 | Smc3 | 0.042122 |
| | binding | | | | |
| 0035255 | ionotropic glutamate | 39 | 2 | Neto1, Pten | 0.043957 |
| | receptor binding | | | | |
| 0047676 | arachidonate-CoA ligase | 6 | 1 | Acsl4 | 0.050333 |
| | activity | | | | |
| 0016907 | G-protein coupled | 6 | 1 | Chrm3 | 0.050333 |
| | acetylcholine receptor | | | | |
| | activity | | | | |
| 0034604 | pyruvate dehydrogenase | 6 | 1 | Dld | 0.050333 |
| | (NAD+) activity | | | | |
| 0035368 | selenocysteine insertion | 6 | 1 | Ncl | 0.050333 |
| | sequence binding | | | | |
| 0019992 | diacylglycerol binding | 6 | 1 | Pltp | 0.050333 |
| 1990050 | phosphatidic acid | 6 | 1 | Pltp | 0.050333 |
| | transporter activity | | | | |
| 1904121 | phosphatidylethanolamin | 6 | 1 | Pltp | 0.050333 |
| | e transporter activity | | | | |
| 0004791 | thioredoxin-disulfide | 6 | 1 | Selenot | 0.050333 |
| | reductase activity | | | | |
| 0005332 | gamma-aminobutyric | 6 | 1 | Slc6a6 | 0.050333 |
| | acid:sodium symporter | | | | |
| | activity | | | | |
| 0004601 | peroxidase activity | 44 | 2 | Hba-a2, Hbb-bs | 0.054596 |
| 0099635 | voltage-gated calcium | 7 | 1 | Cnr1 | 0.058473 |
| | channel activity involved | | | | |
| | in positive regulation of | | | | |
| | presynaptic cytosolic | | | | |
| | calcium levels | | | | |
| 0010385 | double-stranded | 7 | 1 | Egr1 | 0.058473 |
| | methylated DNA binding | | | | |
| 0030911 | TPR domain binding | 7 | 1 | Hsp90aa1 | 0.058473 |
| 1905538 | polysome binding | 7 | 1 | Impact | 0.058473 |
| 1904315 | transmitter-gated ion | 47 | 2 | Gabra2, Gabrb1 | 0.061369 |
| | channel activity involved | | | | |
| | in regulation of | | | | |
| | postsynaptic membrane | | | | |
| | potential | | | | |
| 0061797 | pH-gated chloride | 48 | 2 | Gabra2, Gabrb1 | 0.063687 |
| | channel activity | | | | |
| 0030235 | nitric-oxide synthase | 8 | 1 | Hsp90aa1 | 0.066545 |
| | regulator activity | | | | |
| 0031995 | insulin-like growth factor | 8 | 1 | Igfbp5 | 0.066545 |
| | II binding | | | | |
| 0010997 | anaphase-promoting | 8 | 1 | Pten | 0.066545 |
| | complex binding | | | | |
| 1990247 | N6-methyladenosine- | 8 | 1 | Ythdc1 | 0.066545 |
| | containing RNA binding | | | | |
| 0008028 | monocarboxylic acid | 52 | 2 | Slc6a6, Slco1c1 | 0.073247 |
| | transmembrane | | | | |
| | transporter activity | | | | |
| 0031957 | very long-chain fatty | 9 | 1 | Acsl4 | 0.074547 |
| | acid-CoA ligase activity | | | | |
| 0030957 | Tat protein binding | 9 | 1 | Dnaja1 | 0.074547 |
| 0034046 | poly(G) binding | 9 | 1 | Fmr1 | 0.074547 |
| 0003691 | double-stranded | 9 | 1 | Pura | 0.074547 |
| | telomeric DNA binding | | | | |
| 0005544 | calcium-dependent | 53 | 2 | Anxa5, Cpne7 | 0.075705 |
| | phospholipid binding | | | | |
| 0035197 | siRNA binding | 10 | 1 | Fmr1 | 0.082482 |
| 0045159 | myosin II binding | 10 | 1 | Gsn | 0.082482 |
| 0043208 | glycosphingolipid | 10 | 1 | Mag | 0.082482 |
| | binding | | | | |
| 0008199 | ferric iron binding | 10 | 1 | Trf | 0.082482 |
| 0005388 | calcium-transporting | 11 | 1 | Anxa5 | 0.090349 |
| | ATPase activity | | | | |
| 0004143 | diacylglycerol kinase | 11 | 1 | Dgkb | 0.090349 |
| | activity | | | | |
| 0016274 | protein-arginine N- | 11 | 1 | Fbxo11 | 0.090349 |
| | methyltransferase | | | | |
| | activity | | | | |
| 0008503 | benzodiazepine receptor | 11 | 1 | Gabra2 | 0.090349 |
| | activity | | | | |
| 0008429 | phosphatidylethanolamin | 11 | 1 | Pltp | 0.090349 |
| | e binding | | | | |
| 1901611 | phosphatidylglycerol | 11 | 1 | Pltp | 0.090349 |
| | binding | | | | |
| 0005086 | ARF guanyl-nucleotide | 11 | 1 | Psd3 | 0.090349 |
| | exchange factor activity | | | | |
| 1990459 | transferrin receptor | 11 | 1 | Trf | 0.090349 |
| | binding | | | | |
| 0019829 | inorganic cation- | 59 | 2 | Anxa5, mt-Atp6 | 0.09098 |
| | transporting ATPase | | | | |
| | activity | | | | |
| 0042625 | ATPase coupled ion | 61 | 2 | Anxa5, mt-Atp6 | 0.096257 |
| | transmembrane | | | | |
| | transporter activity | | | | |
| 0000900 | translation repressor | 12 | 1 | Pura | 0.098149 |
| | activity, mRNA | | | | |
| | regulatory element | | | | |
| | binding | | | | |
| 0044548 | S100 protein binding | 12 | 1 | S100a1 | 0.098149 |
| 0042910 | xenobiotic transporter | 12 | 1 | Slc6a6 | 0.098149 |
| | activity | | | | |

| | | Total | DE | | |
|---|---|---|---|---|---|
| GO term | Description | Genes | Genes | DE Genes (Names) | P-values |
| 0043218 | compact myelin | 5 | 4 | Mag, Mbp, Pllp, Pmp22 | 2.639E−07 |
| 0043209 | myelin sheath | 182 | 12 | Cldn11, Cnp, Dld, Gjc2, | 2.427E−05 |
| | | | | Gsn, Hsp90aa1, Hspa5, | |
| | | | | Mag, Mbp, Mog, Plcb1, | |
| | | | | Sucla2 | |
| 0035749 | myelin sheath | 6 | 3 | Cnp, Mag, Pten | 6.830E−05 |
| | adaxonal region | | | | |
| 0000235 | astral microtubule | 8 | 3 | Dynlt3, Map9, Pafah1b1 | 1.869E−04 |
| 0098982 | GABA-ergic | 104 | 8 | Camk4, Cnr1, Gabra2, | 1.951E−04 |
| | synapse | | | Gabrb1, Gabrd, Nrxn1, | |
| | | | | Plcb1, Slc6a6 | |
| 1990015 | ensheathing process | 2 | 2 | Mag, Myoc | 2.329E−04 |
| 0097453 | mesaxon | 2 | 2 | Mag, Myoc | 2.329E−04 |
| 0043197 | dendritic spine | 181 | 10 | Akap5, Arc, Fmr1, Fxr1, | 4.823E−04 |
| | | | | Homer1, Lpar1, Mob4, | |
| | | | | Pten, Slc8a1, Syndig1 | |
| 0043198 | dendritic shaft | 69 | 6 | Akap5, Hcn1, Homer1, | 6.462E−04 |
| | | | | Lpar1, Slc8a1, Syndig1 | |
| 0034663 | endoplasmic | 12 | 3 | Hsp90b1, Hspa5, Sdf2l1 | 7.018E−04 |
| | reticulum | | | | |
| | chaperone complex | | | | |
| 0042824 | MHC class I | 14 | 3 | B2m, H2-D1, H2-K1 | 1.135E−03 |
| | peptide loading | | | | |
| | complex | | | | |
| 0005790 | smooth | 31 | 4 | Dnajc3, Fmr1, Hsp90b1, | 1.214E−03 |
| | endoplasmic | | | Hspa5 | |
| | reticulum | | | | |
| 0043220 | Schmidt-Lanterman | 15 | 3 | Mag, Myoc, Pten | 1.403E−03 |
| | incisure | | | | |
| 1902737 | dendritic | 5 | 2 | Fmr1, Fxr1 | 2.259E−03 |
| | filopodium | | | | |
| 0030139 | endocytic vesicle | 154 | 8 | Gsn, Kif5b, Lpar1, Nrxn1, | 2.573E−03 |
| | | | | Rab8b, Rab9b, Rabep1, | |
| | | | | Trf | |
| 1990712 | HFE-transferrin | 6 | 2 | B2m, Trf | 3.354E−03 |
| | receptor complex | | | | |
| 0031415 | NatA complex | 6 | 2 | Naa15, Naa50 | 3.354E−03 |
| 0001651 | dense fibrillar | 6 | 2 | Ncl, Top1 | 3.354E−03 |
| | component | | | | |
| 0042579 | microbody | 127 | 7 | Acbd5, Acs14, Crot, Idi1, | 3.400E−03 |
| | | | | Pex13, Pnpla8, Rab8b | |
| 0030670 | phagocytic vesicle | 21 | 3 | B2m, H2-D1, H2-K1 | 3.832E−03 |
| | membrane | | | | |
| 0005797 | Golgi medial | 23 | 3 | H2-D1, H2-K1, Yipf6 | 4.990E−03 |
| | cisterna | | | | |
| 0060076 | excitatory synapse | 46 | 4 | Akap5, Homer1, Neto1, | 5.266E−03 |
| | | | | Syndig1 | |
| 0060077 | inhibitory synapse | 24 | 3 | Gabra2, Gad2, Nrxn1 | 5.639E−03 |
| 0005844 | polysome | 47 | 4 | Fmr1, Fxr1, Impact, Upf2 | 5.688E−03 |
| 0030666 | endocytic vesicle | 26 | 3 | B2m, H2-D1, H2-K1 | 7.083E−03 |
| | membrane | | | | |
| 0099524 | postsynaptic | 26 | 3 | Fmr1, Homer1, Pten | 7.083E−03 |
| | cytosol | | | | |
| 0005876 | spindle microtubule | 51 | 4 | Bod1l, Dynlt3, Map9, | 7.600E−03 |
| | | | | Pafah1b1 | |
| 0035748 | myelin sheath | 9 | 2 | Cnp, Myoc | 7.809E−03 |
| | abaxonal region | | | | |
| 0044326 | dendritic spine neck | 9 | 2 | Fmr1, Fxr1 | 7.809E−03 |
| 0005833 | hemoglobin | 9 | 2 | Hba-a2, Hbb-bs | 7.809E−03 |
| | complex | | | | |
| 0051286 | cell tip | 10 | 2 | Rab8b, Trf | 9.663E−03 |
| 0005777 | peroxisome | 119 | 6 | Acbd5, Acsl4, Crot, Idi1, | 9.958E−03 |
| | | | | Pex13, Pnpla8 | |
| 0098845 | postsynaptic | 12 | 2 | Akap5, Arc | 0.0139 |
| | endosome | | | | |
| 0009898 | cytoplasmic side of | 61 | 4 | Akap5, G6pdx, Litaf, Pten | 0.0141 |
| | plasma membrane | | | | |
| 1990707 | nuclear | 1 | 1 | Atrx | 0.0153 |
| | subtelomeric | | | | |
| | heterochromatin | | | | |
| 0030990 | intraciliary | 1 | 1 | Dync2li1 | 0.0153 |
| | transport particle | | | | |
| 0005969 | serine-pyruvate | 1 | 1 | Eea1 | 0.0153 |
| | aminotransferase | | | | |
| | complex | | | | |
| 0071540 | eukaryotic | 1 | 1 | Eif3a | 0.0153 |
| | translation initiation | | | | |
| | factor 3 complex, | | | | |
| | eIF3e | | | | |
| 0016028 | rhabdomere | 1 | 1 | Mertk | 0.0153 |
| 0034678 | integrin alpha8- | 1 | 1 | Npnt | 0.0153 |
| | betal complex | | | | |
| 0005943 | phosphatidylinositol | 1 | 1 | Pik3ca | 0.0153 |
| | 3-kinase complex, | | | | |
| | class IA | | | | |
| 0045239 | tricarboxylic acid | 13 | 2 | Dld, Sucla2 | 0.0163 |
| | cycle enzyme | | | | |
| | complex | | | | |
| 1902711 | GABA-A receptor | 13 | 2 | Gabra2, Gabrb1 | 0.0163 |
| | complex | | | | |
| 0071556 | integral component | 13 | 2 | H2-D1, H2-K1 | 0.0163 |
| | of lumenal side of | | | | |
| | endoplasmic | | | | |
| | reticulum | | | | |
| | membrane | | | | |
| 1990124 | messenger | 14 | 2 | Fmr1, Hnrnpa3 | 0.0188 |
| | ribonucleoprotein | | | | |
| | complex | | | | |
| 0005778 | peroxisomal | 38 | 3 | Pex13, Pnpla8, Rab8b | 0.0201 |
| | membrane | | | | |
| 0032590 | dendrite membrane | 39 | 3 | Akap5, Gabra2, Hcn1 | 0.0215 |
| 0098839 | postsynaptic | 39 | 3 | Arc, Neto1, Syndig1 | 0.0215 |
| | density membrane | | | | |
| 0099522 | region of cytosol | 40 | 3 | Fmr1, Homer1, Pten | 0.0230 |
| 0005753 | mitochondrial | 16 | 2 | mt-Atp6, mt-Atp8 | 0.0243 |
| | proton-transporting | | | | |
| | ATP synthase | | | | |
| | complex | | | | |
| 0099634 | postsynaptic | 41 | 3 | Arc, Neto1, Syndig1 | 0.0246 |
| | specialization | | | | |
| | membrane | | | | |
| 0045178 | basal part of cell | 17 | 2 | Cldn11, Trf | 0.0272 |
| 0033270 | paranode region of | 17 | 2 | Gjc2, Mag | 0.0272 |
| | axon | | | | |
| 0055037 | recycling endosome | 113 | 5 | Akap5, Avl9, Eeal, Mctp1, | 0.0298 |
| | | | | Trf | |
| 0032433 | filopodium tip | 18 | 2 | Fmr1, Fzd3 | 0.0303 |
| 0030140 | trans-Golgi network | 18 | 2 | Gopc, Rab8b | 0.0303 |
| | transport vesicle | | | | |
| 0072563 | endothelial | 2 | 1 | Anxa5 | 0.0303 |
| | microparticle | | | | |
| 0043614 | multi-eIF complex | 2 | 1 | Eif3a | 0.0303 |
| 0032998 | Fc-epsilon receptor | 2 | 1 | Fcer1g | 0.0303 |
| | I complex | | | | |
| 0061202 | clathrin-sculpted | 2 | 1 | Gad2 | 0.0303 |
| | gamma- | | | | |
| | aminobutyric acid | | | | |
| | transport vesicle | | | | |
| | membrane | | | | |
| 0097226 | sperm | 2 | 1 | Hsp90aa1 | 0.0303 |
| | mitochondrial | | | | |
| | sheath | | | | |
| 0098560 | cytoplasmic side of | 2 | 1 | Litaf | 0.0303 |
| | late endosome | | | | |
| | membrane | | | | |
| 0005818 | aster | 2 | 1 | Map9 | 0.0303 |
| 1904423 | dehydrodolichyl | 2 | 1 | Nus1 | 0.0303 |
| | diphosphate | | | | |
| | synthase complex | | | | |
| 0030426 | growth cone | 197 | 7 | Cnr1, Fmr1, Fxr1, | 0.0321 |
| | | | | Hsp90aa1, Kif5b, Nrxn1, | |
| | | | | Pafah1b1 | |
| 0044449 | contractile fiber | 198 | 7 | Anxa5, Fxr1, Homer1, | 0.0328 |
| | part | | | Jph1, Npnt, S100a1, | |
| | | | | Slc8a1 | |
| 0044295 | axonal growth cone | 46 | 3 | Hsp90aa1, Kif5b, Nrxn1 | 0.0331 |
| 0090723 | growth cone part | 19 | 2 | Fmr1, Pafah1b1 | 0.0336 |
| 0043034 | costamere | 19 | 2 | Fxr1, Homer1 | 0.0336 |
| 0005922 | connexin complex | 19 | 2 | Gjb1, Gjc2 | 0.0336 |
| 0043679 | axon terminus | 121 | 5 | Anxa5, Chrm3, Fmr1, | 0.0383 |
| | | | | Hcn1, Slc8a1 | |
| 0045335 | phagocytic vesicle | 83 | 4 | Gsn, Kif5b, Rab8b, Rab9b | 0.0384 |
| 0030018 | Z disc | 124 | 5 | Anxa5, Homer1, Jph1, | 0.0418 |
| | | | | S100a1, Slc8a1 | |
| 0099055 | integral component | 167 | 6 | Chrm3, Gabra2, Gabrd, | 0.0435 |
| | of postsynaptic | | | Neto1, Slc6a6, Slc8a1 | |
| | membrane | | | | |
| 0005921 | gap junction | 22 | 2 | Gjb1, Gjc2 | 0.0440 |
| 0098855 | HCN channel | 3 | 1 | Hcn1 | 0.0452 |
| | complex | | | | |
| 0097524 | sperm plasma | 3 | 1 | Hsp90aa1 | 0.0452 |
| | membrane | | | | |
| 0014701 | junctional | 3 | 1 | Jph1 | 0.0452 |
| | sarcoplasmic | | | | |
| | reticulum | | | | |
| | membrane | | | | |
| 0098559 | cytoplasmic side of | 3 | 1 | Litaf | 0.0452 |
| | early endosome | | | | |
| | membrane | | | | |
| 0034457 | Mpp10 complex | 3 | 1 | Mphosph10 | 0.0452 |
| 1990415 | Pex17p-Pex14p | 3 | 1 | Pex13 | 0.0452 |
| | docking complex | | | | |
| 0042709 | succinate-CoA | 3 | 1 | Sucla2 | 0.0452 |
| | ligase complex | | | | |
| 0035327 | transcriptionally | 23 | 2 | Aff4, Psip1 | 0.0477 |
| | active chromatin | | | | |
| 0031307 | integral component | 24 | 2 | Armcx3, Gdap1 | 0.0515 |
| | of mitochondrial | | | | |
| | outer membrane | | | | |
| 0032279 | asymmetric synapse | 25 | 2 | Akap5, Chrm3 | 0.0555 |
| 0005868 | cytoplasmic dynein | 25 | 2 | Dync2li1, Dynlt3 | 0.0555 |
| | complex | | | | |
| 0032783 | ELL-EAF complex | 4 | 1 | Aff4 | 0.0598 |
| 0043159 | acrosomal matrix | 4 | 1 | Dld | 0.0598 |
| 0044308 | axonal spine | 4 | 1 | Eea1 | 0.0598 |
| 1990812 | growth cone | 4 | 1 | Fmr1 | 0.0598 |
| | filopodium | | | | |
| 0097444 | spine apparatus | 4 | 1 | Fmr1 | 0.0598 |
| 0019034 | viral replication | 4 | 1 | Fmr1 | 0.0598 |
| | complex | | | | |
| 0030478 | actin cap | 4 | 1 | Gsn | 0.0598 |
| 0042567 | insulin-like growth | 4 | 1 | Igfbp5 | 0.0598 |
| | factor ternary | | | | |
| | complex | | | | |
| 0035976 | transcription factor | 4 | 1 | Junb | 0.0598 |
| | AP-1 complex | | | | |
| 0098574 | cytoplasmic side of | 4 | 1 | Litaf | 0.0598 |
| | lysosomal | | | | |
| | membrane | | | | |
| 0033269 | internode region of | 4 | 1 | Mbp | 0.0598 |
| | axon | | | | |
| 0031021 | interphase | 4 | 1 | Mzt1 | 0.0598 |
| | microtubule | | | | |
| | organizing center | | | | |
| 0030289 | protein phosphatase | 4 | 1 | Ppp4r2 | 0.0598 |
| | 4 complex | | | | |
| 0008305 | integrin complex | 28 | 2 | Npnt, Pmp22 | 0.0679 |
| 0098563 | intrinsic component | 63 | 3 | Gabra2, Rab3c, Wfs1 | 0.0719 |
| | of synaptic vesicle | | | | |
| | membrane | | | | |
| 0070971 | endoplasmic | 29 | 2 | H2-D1, H2-K1 | 0.0722 |
| | reticulum exit site | | | | |
| 0031256 | leading edge | 146 | 5 | Akap5, Gabra2, Hcn1, | 0.0737 |
| | membrane | | | Hsp90aa1, Psd3 | |
| 0061673 | mitotic spindle | 5 | 1 | Dynlt3 | 0.0741 |
| | astral microtubule | | | | |
| 0044094 | host cell nuclear | 5 | 1 | Fmr1 | 0.0741 |
| | part | | | | |
| 1990769 | proximal neuron | 5 | 1 | Gjc2 | 0.0741 |
| | projection | | | | |
| 0030485 | smooth muscle | 5 | 1 | Npnt | 0.0741 |
| | contractile fiber | | | | |
| 0016586 | RSC complex | 5 | 1 | Pbrm1 | 0.0741 |
| 0034991 | nuclear meiotic | 5 | 1 | Smc3 | 0.0741 |
| | cohesin complex | | | | |
| 0097433 | dense body | 5 | 1 | Trf | 0.0741 |
| 0098984 | neuron to neuron | 30 | 2 | Akap5, Chrm3 | 0.0766 |
| | synapse | | | | |
| 0030672 | synaptic vesicle | 66 | 3 | Gad2, Mctp1, Syndig1 | 0.0802 |
| | membrane | | | | |
| 0005791 | rough endoplasmic | 67 | 3 | Ccdc47, Clock, Fmr1 | 0.0830 |
| | reticulum | | | | |
| 0005726 | perichromatin | 6 | 1 | Clock | 0.0883 |
| | fibrils | | | | |
| 0031466 | Cul5-RING | 6 | 1 | Cul5 | 0.0883 |
| | ubiquitin ligase | | | | |
| | complex | | | | |
| 0071598 | neuronal | 6 | 1 | Fmr1 | 0.0883 |
| | ribonucleoprotein | | | | |
| | granule | | | | |
| 0008274 | gamma-tubulin ring | 6 | 1 | Mzt1 | 0.0883 |
| | complex | | | | |
| 0090724 | central region of | 6 | 1 | Pafah1b1 | 0.0883 |
| | growth cone | | | | |
| 0000932 | cytoplasmic mRNA | 72 | 3 | Dcp2, Pnrc2, Top1 | 0.0979 |
| | processing body | | | | |
| 0032040 | small-subunit | 35 | 2 | Krr1, Mphosph10 | 0.0997 |
| | processome | | | | |

The most significantly affected canonical pathways after exposure included protein ubiquitination (B2m, Dnaja1, Dnajb4, Hba-a2, Hsp90aa1, Hsp90b1, Hspa41, Hspa5, Ube2q2), aldosterone signaling in epithelial cells (Dnaja1, Dnajb4, Hsp90aa1, Hsp90b1, Hspa41, Hspa5, Plcb1), hypoxia signaling in the cardiovascular system (Hsp90aa1, Hsp90b1, Nfkbia, Pten, Ube2q2), unfolded protein response (Hsp90b1, Hspa5, Srebf1), endoplasmic reticulum (ER) stress pathway (Hsp90b1, Hspa5), and the neuroinflammation signaling pathway (B2m, Gabra2, Gabrb1, Gad2, Hba-a2).

Dysregulation of genes indicative of a pro-inflammatory response, including downregulation of B2m and Hba-a2 and upregulation of Gabra2, Gabrb1, and Gad was also observed. There was significant downregulation of several genes associated with neuronal health, particularly genes involved in the integrity of the myelin sheath (Mog, Mbp, Mag, PUP, Pmp22, Cldn11, Cnp), neurogenesis (Arc, Opalin), dendritic cell maturation (B2m, Hba-a2), NF-κB inhibition (Nfkbia, Plcb1), and learning and memory (Arc). Additionally, significant downregulation of mitochondrial genes coding for the F0 subunit of the proton-transporting ATP-synthase complex (mt-Atp6, mt-Atp8) was also found. There was significant upregulation of pro-apoptotic genes (Pten), genes involved in ER stress response (Hspa5, Hsp90b1), and genes involved in organonitrogen compound metabolism (Lars2, Hmgn5). There was also upregulation of genes implicated in related neurodegenerative diseases, including Oxr1, Top1, and Cdr1.

Dysregulation in gene ontology categories of interest relating to biological processes, molecular functions, and cellular components was also observed. Significantly affected biological processes included leucyl-tRNA aminoacylation (Lars2), regulation of neurogenesis (Arc, Opalin), peptide metabolic process (Hmgn5, Lars2), regulation of long-term synaptic depression (Arc), regulation of postsynaptic neurotransmitter receptor internalization (Arc), and mitochondrial translation (Lars2). Notably affected GO categories involved in molecular functions included RNA strand annealing activity (Fmr1, Fxr1), supercoiled DNA binding (Psip1, Top1), and unfolded protein binding (Dnajb4, Hsp90aa1, Hsp90b1, Hspa5). Significantly affected gene ontology categories forming cellular components of interest included the myelin sheath (Cldn11, Cnp, Dld, Gjc2, Gsn, Hsp90aa1, Hspa5, Mag, Mbp, Mog, Plcb1, Sucla2), GABAergic synapses (Camk4, Cnr1, Gabra2, Gabrb1, Gabrd, Nrxn1, Plcb1, Slc6a6), dendritic spines (Akap5, Arc, Fmr, Fxr1, Homer1, Lpar1, Mob4, Pten, Slc8a1, Syndig1), endoplasmic reticulum chaperone complex (Hsp90b1, Hspa5, Sdf2l1), MHC class I peptide loading complex (B2m, H2-D1, H2-K1), and endocytic vesicles (Gsn, Kif5b, Lpar1, Nrxn1, Rab8b, Rab9b, Rabep1, Trf), among others.

Discussion. The results showed that subcutaneous administration of PB+CPF+DEET for two weeks induced acute changes in gene expression in mouse hippocampal tissue, including dysregulation of genes indicating a pro-inflammatory response, downregulation of genes associated with neuronal health, and upregulation of pro-apoptotic genes, genes involved in ER stress response, and genes implicated in neurogenerative diseases, among others. Significant effects of the Gulf War exposure on spatial memory was also observed.

The three most significantly downregulated genes after exposure were Arc, Egr1, and Nr4a1, of which are neuronal immediate early genes (IEGs). Arc is predominantly expressed in cortical and hippocampal glutamatergic neurons and is involved in numerous neuronal signaling pathways (I. Epstein, S. Finkbeiner, Semin Cell Dev Biol 77 (2018) 63-72; and E. Korb, S. Finkbeiner, Trends Neurosci 34 (2011) 591-598). Arc knockout mice display deficits in long-term memory formation in implicit and explicit learning tasks and impaired long-term potentiation (LTP) and depression (LTD) (N. Plath, et al., Neuron 52 (2006) 437-444); similar effects on LTP and spatial learning were shown in rats after chemical inhibition of Arc (J. F. Guzowski, et al., J Neurosci 20 (2000) 3993-4001). Egr1 is required for stabilization of synaptic plasticity in the hippocampus as well as formation of both hippocampal and non-hippocampal-dependent long-term memory (M. W. Jones, et al., Nat Neurosci 4 (2001) 289-296) and is a direct transcriptional regulator of Arc (L. Li, et al., Mol Cell Biol 25 (2005) 10286-10300).

Although IEGs are classified as such due to their early and transient response to environmental stimuli, both Arc and Egr1 also play important roles in mediating the structural changes that underlie neuronal and synaptic plasticity, showing that their dysregulation can trigger long-term morphological changes with negative impacts on learning and memory formation. Several mouse models of Alzheimer's disease report early dysregulation of IEGs involved in LTP and synaptic plasticity (J. N. Perusini, et al., Hippocampus 27 (2017) 1110-1122). Dickey et al. observed a significant decrease in basal Arc, Egr1, and Nr4a1 expression in amyloid-containing hippocampus and cortex of APP/PS1 transgenic mice (C. A. Dickey, et al., J Neurosci 23 (2003) 5219-5226). Levels of basal and exploration-induced Arc expression are significantly reduced in granule cells of the dentate gyrus of hAPP_{FAD} transgenic mice (J. J. Palop, et al., J Neurosci 25 (2005) 9686-9693). Induced Arc expression was also dysregulated in the CA3 region and dentate gyrus of rats chronically infused with lipopolysaccharide (LPS) to induce neuroinflammation, suggesting altered patterns of Arc expression may contribute to cognitive and memory impairments in neurodegeneration (S. Rosi, et al., J Neurosci 25 (2005) 723-731).

IEGs such as Arc and Egr1 have also been suggested to play an important role in the interaction between genes and environment to determine the risk of developing psychiatric illness, particularly major depressive disorder (MDD), which is typically comorbid with GWI (Institute of Medicine, Gulf War and Health: Volume 8: Update of Health Effects of Serving in the Gulf War, Washington, D.C., 2010; Institute of Medicine, Chronic Multisymptom Illness in Gulf War Veterans: Case Definitions Reexamined, Washington, D.C., 2014; United States Department of Veterans Affairs, Research Advisory Committee on Gulf War Veterans' Illnesses [RAC-GWI], Gulf War illness and the health of Gulf War veterans: scientific findings and recommendations, Washington, D.C., 2008; Institute of Medicine, Gulf War Veterans: Treating Symptoms and Syndromes, Washington, D.C., 2001; Institute of Medicine, Gulf War and Health: Treatment for Chronic Multisymptom Illness, Washington, D.C., 2013; L. Steele, Am J Epidemiol 152 (2000) 992-1002; R. F. White, et al., Cortex 74 (2016) 449-475; A. L. Gallitano, Front Behav Neurosci 14 (2020) 16; F. Duclot, M. Kabbaj, Front Behav Neurosci 11 (2017) 35; Y. Xu, et al, Neurobiol Aging 36 (2015) 955-970; H. E. Covington, 3rd, et al., J Neurosci 30 (2010) 16082-16090). Chronic treatment with various antidepressants targeting serotonin and norepinephrine can also restore Arc expression in the hippocampus and prefrontal cortex (F. T. Gallo, et al., Front Behav Neurosci 12 (2018) 79; and Y. Li, et al., Front Neurosci 9 (2015) 279).

Additionally, Arc inhibits the binding of heat shock factor 1 (HSF1) to the heat shock element (HSE) in heat shock protein (HSP) gene promoters and prevents activation of HSP genes (A. Y. Park, et al., Sci Rep 9 (2019) 2592). Accordingly, upregulation of HSP genes, including Hsp40s (Dnajb4, Dnaja1), Hsp70s (Hspa41, Hspa5), and Hsp90s (Hsap90aa1, Hsp90b1) was observed and it was found that these genes were involved in several significantly affected pathways, including protein ubiquitination, aldosterone signaling, hypoxia signaling, unfolded protein response, interferon induction and antiviral response, and the ER stress pathway, among others. Thus, dysregulation of IEGs may play a role in acute neuroinflammation, leading to chronic neurodegeneration.

Interestingly, several genes encoding proteins that are structural components of myelin were downregulated, including Mbp, Mag, Mog, and Cnp. Myelin basic protein (Mbp) is phosphorylated by MAP kinase in response to action potential firing during LTP in the hippocampus (C. M. Atkins, et al., J Neurochem 73 (1999) 1090-1097; and P. R. Lee, R. D. Fields, Front Neuroanat 3 (2009) 4). Plasma autoantibodies against Mbp have also been found to be significantly increased in Veterans with symptoms of GWI compared to healthy controls (M. B. Abou-Donia, et al., Neurotoxicol Teratol 61 (2017) 36-46; and M. B. Abou-Donia, et al., Brain Sci 10 (2020)). Dysregulation of genes related to the GABAergic synapse, including Camk4, Cnr1, Gabra2, Gabrb1, Gabrd, Nrxn1, Plcb1, and Slc6a6 were also observed. Chronically, decreased GABA has been reported in hippocampi of mice exposed to PB+permethrin+DEET three months after exposure (I. Carreras, et al., Brain Res 1681 (2018) 34-43). Additionally, decreased expression of Chrm3, which codes for the M₃ muscarinic receptor, was found. Decreased M₃ receptor density has been reported in the CA1 region, CA3 region, and molecular layer of the hippocampus in C57Bl/6 mice exposed to PB+stress (B. Mauck, et al., Neurotoxicology 31 (2010) 461-467). This shows that changes in GABA_{A} and M₃ receptor expression can begin during the acute phase of chronic sublethal exposure to the Gulf War toxicants described herein.

Reported dosages and routes of administration of Gulf War toxicants in rodent models have varied widely throughout the literature. The subcutaneous route of administration for exposure to PB+CPF+DEET has several advantages. PB was taken orally by military personnel and is frequently administered via gavage in animal models; however, PB has been shown to have poor bioavailability, suggesting that injection may deliver a more precise dosage (L. Abdullah, et al., Neuromolecular Med 13 (2011) 275-288). There has also been a significant amount of investigation into the effects of stress in combination with PB and other toxicants, with results that indicate increased BBB permeability to toxicants in stressed animals (A. Abdel-Rahman, et al., Neurobiol Dis 10 (2002) 306-326). Friedman et al. reported significant effects of PB+stress on levels of c-Fos and AChE mRNAs in mouse whole-brain homogenates, indicating that stress can be a confounding variable in gene expression data examining an early transcriptional response (A. Friedman, et al., Nat Med 2 (1996) 1382-1385). The subcutaneous route would not present potential stress from repeated oral gavage.

Subcutaneous administration also avoids variable absorption via dermal application of CPF and DEET, which would have been in contact with the skin of military personnel. A study by Keil et al. examining the immunotoxicology of DEET in female B6C3F1 mice elaborated on factors which are important to accurately compare exposures in animal models but are often not considered (D. E. Keil, et al., Toxicol Sci 108 (2009) 110-123). Many human and animal studies refer to dermal penetration rather than absorption into the bloodstream, which is not an equivalent measure due to the variability of absorption levels within and between species. Keil et al. reported that s.c. administration of 7.7 mg/kg/day DEET equates to an estimated mouse blood exposure level that encompasses estimated military exposure levels as well as estimated DEET usage by the general population. Additionally, Keil et al. argue that the emphasis placed on relevant route of exposure in the literature has limited utility, particularly in the case of dermal exposures such as DEET or CPF. CPF, a lipophilic organophosphate, could accumulate within the brain to cause AChE inhibition at the acute timepoint, which could have an effect on behavioral outcomes. There are wide ranges of estimated absorption and metabolic rates between rodents and humans.

It should be noted that military personnel would have been exposed to these compounds at lower dosages, but this exposure occurred over longer periods of time. In rodent models, higher dosages are often used in a shorter time frame due to the lifespan of the animal and the window in which to study effects. Other studies have reported using similar dosages at these intervals: Lamproglou et al. reported i.o. administration of 1.5 mg/kg PB for 12 days (5 days on, 2 days off, 5 days on) in male Wistar rats (I. Lamproglou, et al., Behav Brain Res 197 (2009) 301-310); Peden-Adams et al. treated female B6C3F1 mice treated with 15.5 mg/kg DEET, 2 mg/kg PB, and 500 mg/kg JP-8 s.c. for 14 days as a “low dose” group (M. M. Peden-Adam, et al., Toxicol Ind Health 17 (2001) 192-209); Torres-Altoro et al. reported treatment of female C57Bl/6 mice with 30 mg/kg CPF s.c. for 7 days, male FVB mice with 2.5 mg/kg PB+5 mg/kg DEET s.c. for 15 days, and male C57Bl/6 treated with 1 mg/kg PB s.c. for 10 days (M. I. Torres-Altoro, et al., J Neurochem 119 (2011) 303-313)); and Mauck et al. treated male C57Bl/6 mice with 3 or 10 mg/kg PB for 7 days via s.c. ALZET pump (B. Mauck, et al., Neurotoxicology 31 (2010) 461-467). These studies illustrate the similar range of concentrations over shorter time frames, as well as the advantages of s.c. administration for certain experiments.

Whole transcriptome sequencing has been used in several rodent models of Gulf War exposure. A similar study by Shetty et al. examined changes in gene expression using qRT-PCR after 4 weeks of exposure to PB+DEET+stress in male Sprague-Dawley rats; however, their samples, collected at a longer 6-month time point after the last exposure, presented a gene expression profile indicative of chronic neuroinflammation (G. A. Shetty, et al., Front Mol Neurosci 10 (2017) 182). Gene expression profiles of GWI patients have also been studied to identify treatment strategies by examining the overlap of dysregulated genes with drug targets and comparison to expression profiles of other diseases (T. J. Craddock, et al., BMC Med Genomics 8 (2015) 36). In contrast, the acute Gulf War exposure model described herein shows early effects that do not appear in chronic exposure models, such as dysregulation of IEGs. Xu et al. also recently reported on acute transcriptional changes in BXD mouse strains after exposure to corticosterone+diisopropyl fluorophosphate (DFP) (F. Xu, et al., Brain Behav Immun 89 (2020) 209-223).

Conclusion. This study provides an assessment of changes in gene expression in combined exposure to PB, CPF, and DEET and a gene expression profile at an acute time point. Many of the dysregulated genes involve inflammatory signaling and other pathways that are important for the health of neurons. The neurological effects of toxicants, including memory deficits, may begin soon after exposure.

1. A method of treating of Gulf War illness or syndrome in a subject, the method comprising: administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist.

2. The method of claim 1, wherein the PPAR-γ agonist is pioglitazone or rosiglitazone.

3. The method of claim 1, wherein the Nrf2 agonist is tert-butylhydroquinone (t-BHQ) or sulforaphane.

4. (canceled)

5. (canceled)

6. The method of claim 2, wherein the pioglitazone is administered orally, subcutaneously or intraperitoneally.

7. The method of claim 3, wherein the t-HBQ is administered orally, subcutaneously or intraperitoneally.

8. The method of claim 2, wherein the therapeutically effective amount of pioglitazone is 0.1 mg to 0.4 mg/kg body weight per day.

9. The method of claim 3, wherein the therapeutically effective amount of t-BHQ is 1.0 mg to 5.0 mg/kg body weight per day.

10. The method of claim 1, further comprising administering a therapeutically effective amount of one or more transcription factor modulators.

11. The method of claim 3, wherein the administration of pioglitazone and t-BHQ reduces or ameliorates one or more symptoms of Gulf War illness or syndrome.

12. The method of claim 11, wherein the one or more symptoms of Gulf War illness or syndrome is fatigue, musculoskeletal pain, skin rashes, diarrhea, headache, memory loss, spatial memory deficits, sleep disturbances or a combination thereof.

13. The method of claim 1, wherein the administration of PPAR-γ) agonist and a nuclear factor erythroid 2-related factor 2 (Nrf2) agonist increases stamina, improve cognition, improve information seeking or a combination thereof in the subject.

14. A method of ameliorating one or more symptoms of ameliorating one or more symptoms of Gulf War illness or syndrome in a subject, the method comprising: administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist.

15. A method of inhibiting neurodegeneration or effecting neuroprotection in a subject in need thereof, the method comprising: administering to the subject in need thereof a therapeutically effective amount of a nuclear receptor peroxisome proliferator-activated receptor gamma (PPAR-γ) agonist and a nuclear factor erythroid 2-related factor 2 (Nrf2) agonist.

16. The method of claim 15, wherein the neurodegeneration or neuroprotection is associated with Alzheimer's disease, Parkinson's disease, traumatic brain injury, amyotrophic lateral sclerosis, ischemic stroke or a combination thereof.

17. The method of claim 14, wherein the PPAR-γ agonist is pioglitazone.

18. The method of claim 14, wherein the Nrf2 agonist is tert-butylhydroquinone (t-BHQ).

19. (canceled)

20. (canceled)

21. The method of claim 17, wherein the pioglitazone is administered orally.

22. The method of claim 18, wherein the t-HBQ is administered orally.

23. The method of claim 17, wherein the therapeutically effective amount of pioglitazone is 0.1 mg to 0.4 mg/kg body weight per day.

24. The method of claim 18, wherein the therapeutically effective amount of t-BHQ is 1.0 mg to 5.0 mg/kg body weight per day.

25.-31. (canceled)
